(19) United States
(12) Patent Application Publication
(10) Pub. No.: US 2020/0397936 A1
(43) Pub. Date: Dec. 24, 2020

(54) Systems and Methods For Internet-Of-Things (IOT) Robotic Sterilization Device

(71) Applicant: ATOM, Inc., Scottsdale, AZ (US)

(72) Inventors: Yani Deros, Phoenix, AZ (US); Jodi Deros, Phoenix, AZ (US)

(21) Appl. No.: 17/001,587

(22) Filed: Aug. 24, 2020

Related U.S. Application Data
(63) Continuation-in-part of application No. 16/418,935, filed on May 21, 2019.

(51) Int. Cl. A61L 2/24; G16Y 40/10; G16Y 10/80; B25J 9/16; G05B 19/042; G05B 19/4155; A61L 2/22; A61L 2/10; F24F 3/16; F24F 11/58; G16Y 10/60

(52) U.S. Cl. CPC A61L 2/24; G16Y 40/10; G16Y 10/80; B25J 9/1689; G05B 19/042; G05B 19/4155; A61L 2101/20; A61L 2/10; F24F 3/16; F24F 11/58; G16Y 10/60; G05B 2219/2614; G05B 2219/50391; A61L 2/22

(57) ABSTRACT

An internet-of-things (IOT) robotic sterilization system that operates autonomously for use in the prevention of diseases, e.g., Coronavirus Disease 2019 (COVID-19), caused by pathogens such as coronavirus SARS-CoV-2 and other pathogens present within an interior space is described. A robotic sterilization device is communicatively coupled to the IOT base module via an IOT network, and includes a misting or fogging system fluidically coupled to a liquid reservoir, a sensor module including plurality of sensors, a controller, and a locomotion system. The robotic sterilization device navigates a path within the interior space while creating a disinfecting mist with the misting system, and may coordinate with other IOT-connected devices, such as the HVAC system, UV Vent sterilizers, scent dispensing appliances, and others to more efficaciously sanitize the interior space to protect humans by eliminating pathogens.

### CROSS-REFERENCE TO RELATED APPLICATIONS

This application claims priority to, and is a continuation-in-part of, U.S. patent application Ser. No. 16/418,935, filed May 21, 2019.

### TECHNICAL FIELD

The present invention generally relates to products and processes for use in the prevention and/or treatment of microorganisms and diseases within an interior space, such as Coronavirus Disease 2019 (COVID-19), known to be caused by the novel coronavirus SARS-CoV-2. More particularly, the present invention relates to automated systems and methods for sanitizing, sterilizing, or otherwise treating hospitals, hotel rooms, schools, commercial spaces, and similar interior spaces to reduce or eliminate viruses, bacteria, and other harmful pathogens, thereby autonomously reducing exposure to humans and reducing the corresponding biothreat.

### BACKGROUND

The COVID-19 pandemic of 2020 continues to pose one of the most devastating global health challenges of the last century, causing global disruption of nearly all health, economic, and social systems. The pandemic has reshaped society around the world in a number of respects, and its long-term effects will be felt in permanent changes to people's practices, routines, behaviors and expectations surrounding all human activities, including commerce, employment, education, child-care, travel, entertainment, and all activities involving a work, travel, education and almost every other public setting or gathering.

Many of these societal changes are driven by, or accompanied by, ongoing lockdowns and continued fear public of venturing into public spaces with a great deal of uncertainty surrounding virus transmission as well as best practices for cleaning, sterilization, and otherwise treating spaces and areas in order to reduce exposure risks. This has driven an increased interest in migrating hotel rooms, businesses, and other public spaces toward “touchless” processes that minimize the need for physical interaction with objects in an environment.

In the context of hotel rooms, for example, many establishments are incorporating touchless systems, such as voice control, check in kiosks, wireless point-of-sale devices, bathroom water proximity sensors, wireless door locks, and the like. A recent poll of hotel guests that surveyed what specific actions or process improvement are required to persuade individuals to return to hotels again, with respect to guest expectations, “more intense room cleaning” was the number one spot on the list. Additionally, the respondents also stated as a guest they expect the hotel to keep the room vacant for up to 48 hours in between guest stays to minimize the cross contamination and pathogen exposure possibility.

Unfortunately, room cleaning and sterilization continues to be a costly and labor-intensive process when conforming to new COVID-19 mitigation cleaning protocols, requiring substantial downtime (48 hours, or more), generally workers are using traditional manual hand cleaning methods with gloves and cloths where they are scrubbing with harsh chemicals, adding additional time and steps in the process that add to costly staff training and expenses. Recent data highlights that the increased cleaning supplies, labor and protocol has increased cleaning costs by upwards of 40 percent and require additional trained workers to handle the extended cleaning protocols and burdens. A study published in March 2020 involving monitoring over five and a half weeks showed that overall, only half of all surfaces were adequately cleaned during the study period. The study furthermore concluded that, “despite our best efforts, 100 percent cleaning and disinfection is unlikely to occur. This is important to remember, as regardless of where you visit, it's also best to assume surfaces may be contaminated—and before you come back into your home, you should follow the recommendations to clean your hands and clean items you've handled.”

Non-hospital properties including hotels, office spaces, schools and etc will need to have janitorial workers and even staff be burdened with the responsibility to enhance their cleaning protocols to hospital level standards. Yet with these new higher standard cleaning protocols being enacted across the country studies from hospitals where staff are highly trained for elevated standards of cleaning and sterilization to keep patients from being contracting pathogens shows that up to 40 percent of all healthcare-associated infections are from unclean surfaces and the hands of healthcare workers (Weber, 2010). Research also indicates that only 34 percent to 40 percent of hospital surfaces are cleaned to policy standards (Carting, 2010). This data reinforces that manual cleaning practices has been shown to miss in excess of fifty percent of surfaces, which will lead to transmission and infections by unsterilized areas, including contact surfaces. Cleaning environmental areas and surfaces must have thorough, redundant, back-up protection, reducing the risk that pathogens will not be spread when more “direct” precautions fail. Advocates recommend sanitizing and sterilization system should have in place at least two processes on any potential route that are capable of preventing spread. Two sanitizing processes that are each 50% effective ill produce an overall effect of over 70%. This approach acknowledges that failures will occur in any sanitizing process that is manually carried out. If two such processes are in place, a failure in one will, in most cases, be compensated for by the second. Furthermore, when housekeeping enters a guest room, they are at risk of being exposed to a guest that may have deposited and contaminated the space with pathogens. Another key element is that in some cases the housekeeper may deposit or bring in the pathogens into the space, thus increasing the potential of leaving pathogens after they complete the room cleaning and inadvertently contaminating the space and exposing the guest to the pathogens.

Additionally, in schools where there are typically just a few custodians or janitors for hundreds of rooms, the Center for Disease Control has issued new guidance as of Jul. 25, 2020 that specifies that “cleaning staff should clean and disinfect frequently touched surfaces at least once a day, or more frequently if possible.” Further guidance provides: “Intensify cleaning and disinfection by cleaning staff. Frequently touched surfaces should be cleaned and disinfected at least once a day (i.e., before or after school day), and more frequently when possible. Railings, desks and tables, door and window handles, sanitation (restroom/toilet/latrine) surfaces, toys, teaching/learning aids, and materials used/shared by students (e.g., pens, pencils, art supplies, books, electronics) are examples of frequently touched surfaces.”

Additional details include: “If schools use an expanded timetable (e.g., one group of students attends in the morning and another in the afternoon and/or evening), cleaning and disinfection must occur between each session. This should be extended if any school has a full day of classes that in between each class session the rooms and spaces should be cleaned and sterilized. Many school districts are requiring teachers to purchase their own cleaning supplies and personally clean and sanitize their classrooms in between classes bearing the full burden of implementing, monitoring and maintaining thorough cleaning protocols during the day to protect their students and themselves from exposure to pathogens. This is a burdensome practice for teachers that is not a practical or effective use of resources or time that will lead to missed areas and inconsistent manual cleaning of surfaces. In most instances schools are enacting deep cleaning and sterilization of all classrooms and spaces after school ends each day which does little to nothing to stop the spread of pathogens during school where each person can be exposed and infected during the course of the daily activities and well before afterhours enhanced cleaning and sanitizing procedures to prepare for the next day.”

While some powered disinfecting and misting systems have been employed, particularly in the hospital context, such systems—including standalone UV stations, industrial handheld misters or carts that need to be moved from room to room manually, and the like—tend to be expensive or unavailable due to the back log of orders. Furthermore, such hand-held systems require manual labor to sanitize a room or space, which can lead to misuse or missed surfaces due to human error and/or lapses due to the repetitive and mundane nature of the activity. Generally, when large carts or systems are employed that remain stationary during treatments, these systems require workers entering into rooms or spaces, which may cause them to be infected with pathogens while the systems are manually moved from space to space. The majority rely on manual labor to implement the cleaning processes or migrate from space to space where they cannot adapt to changing environments. In contrast to the prior art, it would be preferable for a machine to do controlled and the programmed work of sterilizing spaces, thus reducing exposure of humans to infected rooms and areas with pathogens. Finally, known prior art misting systems cannot coordinate with any air handlers, air-conditioning units, and other HVAC components that might affect airflow in the room being sanitized, greatly reducing the efficacy of such systems.

Accordingly, there is a long-felt need for smart, automated, and adaptive products and processes for use in sterilizing interior spaces in a way that prevents diseases caused by viruses and other pathogens.

### BRIEF SUMMARY

To achieve the foregoing and other objectives in accordance with the present invention as broadly described herein, a touchless, smart, internet-of-things (IOT) robotic sterilization device—coordinating with an IOT hub or base unit—is permanently installed into each room or space or has the ability to autonomously navigate to and from each space for treatment. The device is configured to navigate through a space in which it is deployed, and spray, fog, or otherwise dispense (e.g., through electrostatic misting) a disinfectant or other beneficial chemical agent (e.g., hypochlorous acid) to kill pathogens that are present on surfaces or remain airborne within the environment.

The use of a IOT hub unit in conjunction with the sterilizing robot is particularly synergistic, as the IOT hub (or other devices within the IOT network) can, for example, through integrated occupancy sensors or remote occupancy sensors, determine if there are humans present in a space. The system can, through Artificial intelligence or scheduled routine via cloud or wireless controls, initiate and complete sterilization of a room and space. The integration of occupancy or remote IoT sensors are essential to reduce exposure of humans to cleaning solutions that may be an irritant. Additionally, the IOT hub has the ability to selectably disable the HVAC system (to prevent premature venting of the disinfectant during the sterilization process) and remotely lock the door of the room (to prevent the entry of personnel during the sterilization process). In some embodiments, this is a timed process that would control both the prescribed and required contact time of the sterilization solution on surfaces and reduce exposure of the solution to humans.

In a particular embodiment, the IOT robotic sterilization device either permanently installed in each space or can navigate between rooms and spaces has an integrated reservoir where it can transport a sterilization/cleaning liquid that is automatically atomized and deployed using an electrostatic misting, ultrasonic humidification with fan or similar method to dispense a cloud of sterilization liquid such as hypochlorous acid. This has been demonstrated as effective in eliminating other human coronavirus strains such as SARS, Noro Virus. See, e.g., Michael S. Block, D M D et al., “Hypochlorous Acid: A Review,” J. Oral Max. Surg., Jun. 25, 2020 (concluding that “[hypochlorous acid] can be used with a high predictability for disinfecting against the COVID-19”). In addition, hypochlorous acid is an all-natural, inexpensive, non-corrosive, colorless, odorless, safe to humans, and environmentally friendly solution.

### BRIEF DESCRIPTION OF THE DRAWING FIGURES

These and other features, aspects, and advantages of the present invention will become better understood with reference to the following description, appended claims, and accompanying drawings, where:

FIG. 1 is a schematic diagram of an enterprise level system including an IOT module, an associated robotic sterilization device, and an API configured to augment tracking data with contextual awareness in accordance with various embodiments;

FIG. 2 is a schematic diagram of an example hotel property illustrating tracking data for a hotel guest within the boundary of the hotel property in accordance with various embodiments:

FIG. 3 is a schematic diagram illustrating tracking data for a hotel guest within and outside the boundary of the hotel property with cellular or area Wi-Fi in accordance with various embodiments:

FIG. 4 is a schematic diagram illustrating the use of beacons in addition to and/or in lieu of traditional GPS based location services in accordance with various embodiments,

FIG. 5 is a schematic diagram of an exemplary in room IOT network that is part of a unified PaaS system including a base module and a plurality of edge devices (e.g., a robotic sterilization system) in accordance with various embodiments;

FIG. 6 is a schematic diagram illustrating a mobile app operating on a mobile device and controlling a plurality of IOT devices in accordance with various embodiments,

FIG. 7 is a more detailed view of the base module in accordance with various embodiments:

FIG. 8 is a schematic view of the stackable electronic hub modules shown in FIG. 7 in accordance with various embodiments:

FIG. 9 is a schematic view of a base module disposed between two beds in a typical hotel, resort, or time share environment in accordance with various embodiments:

FIG. 10 is a schematic view of a base module disposed bedside, illustrating a smart phone charging station in accordance with various embodiments;

FIG. 11 is a schematic view of an alternative embodiment of a base module, illustrating a hotel employee addressing a guest by name based on real time location tracking in accordance with various embodiments delivering a more welcoming and personalized experience;

FIG. 12 is a schematic view of an alternative embodiment of a base module illustrating a self-check-in and check-out system in accordance with various embodiments:

FIG. 13 is a screen display of a mobile app operating on a smart phone illustrating targeted marketing vectors in accordance with various embodiments;

FIG. 14 is a schematic diagram of an in-room IOT module configured to wirelessly communicate with a relay which replaces a conventional wall-mounted thermostat to thereby control an in-room heating, ventilation, and air conditioning (HVAC) unit such as a packaged terminal air conditioner (PTAC) in accordance with various embodiments:

FIG. 15 is schematic diagram of an in-room IOT module configured to wirelessly communicate with a secondary wireless module which wirelessly controls a PTAC or any other HVAC unit in accordance with various embodiments;

FIG. 16 is a schematic diagram of an in-room IOT module wirelessly coupled to a plurality of distributed sensors for monitoring motion and thermal zones in accordance with various embodiments;

FIG. 17 is a conceptual block diagram of an exemplary IOT robotic sterilization device and associated docking station in accordance with one embodiment;

FIGS. 18, 19, and 20 are isometric, close-up, and bottom views, respectively, of the IOT robotic sterilization device of FIG. 17;

FIG. 21 is a cut-away, isometric view of an example room showing the example path that a robotic sterilization device may take during a sterilization session,

FIGS. 22A-E illustrate various alternate designs for a robotic sterilization device with charging bases in accordance with the present invention;

FIGS. 23A and 23B illustrate the use of an internal biosensor that would detect the presence of aerosolized pathogens to determine when that the robotic sterilization device should begin operation or when the there is no longer presence of aerosolized pathogens to complete its monitoring and sterilization process (i.e., fogging or otherwise dispensing a liquid or vapor);

FIG. 24 illustrates the interior of a room with associated biosensor module, ventilation, thermostat, and IOT hub;

FIG. 25 illustrates a variation of the room interior shown in FIG. 24, in which the biosensor module is integrated into the IOT hub;

FIG. 26 is a flowchart depicting a sterilization method in accordance with an exemplary embodiment;

FIGS. 27A and 27B illustrate the use of a docking/refill station in accordance with one embodiment;

FIGS. 28, 29A, 29B, and 30 illustrate example spray patterns in accordance with various embodiments; and

FIGS. 31 and 32 illustrate, respectively, top and three-quarter views of a robot characterized by dual functionality including a misting feature in combination with at least one UV Light integrated into the body of the device to irradiate the immediate space being treated for added efficacy in eliminating pathogens.

FIG. 33 illustrates a wall mounted IOT Hub that includes a display to communicate the room sterilization process along with integrated occupancy sensors (dotted rectangle) that will determine if any humans are present in the room that is being sterilized by the IoT device.

### DETAILED DESCRIPTION OF PREFERRED

### Exemplary Embodiments

The present subject matter generally relates to products and processes for use in the prevention and/or treatment of air, surfaces and areas for pathogens including diseases, such as Coronavirus Disease 2019 (COVID-19). In accordance with various embodiments, an internet-of-things (IOT) robotic sterilization device—coordinating with an IOT hub or base unit—is configured to navigate through the space in which it is operating to disperse and/or spray, atomized fog, or otherwise dispense (e.g., through electrostatic misting) a disinfectant or other beneficial chemical agent (e.g., hypochlorous acid) to kill pathogens such as viruses, spores and/or bacteria that are present on surfaces or remain airborne within the environment.

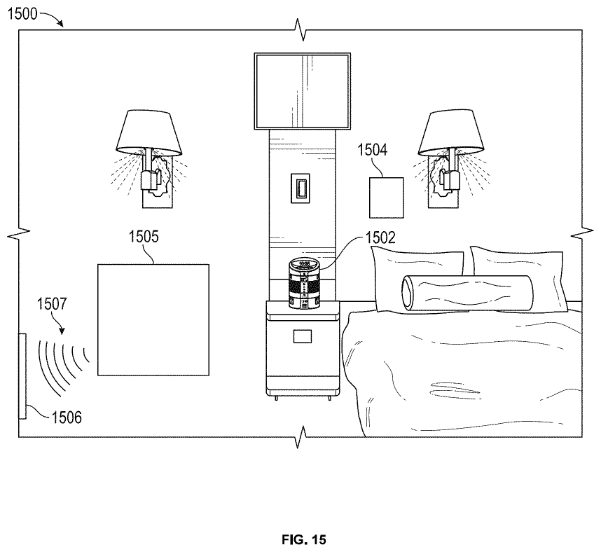

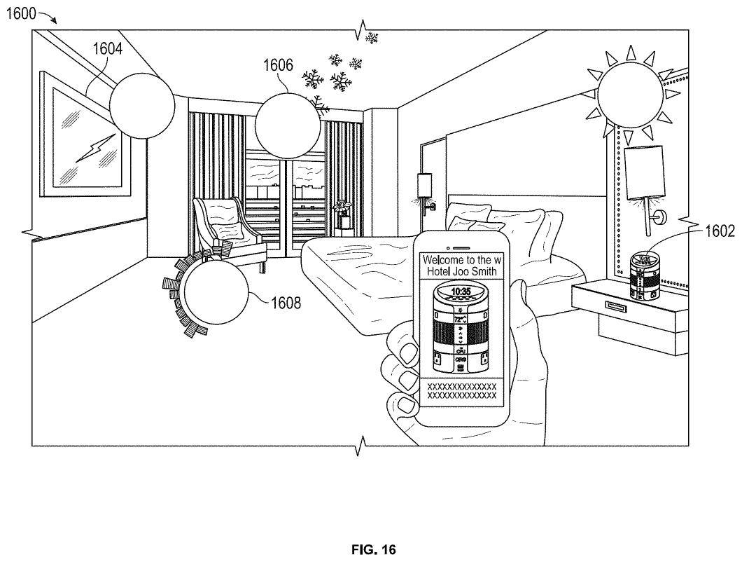

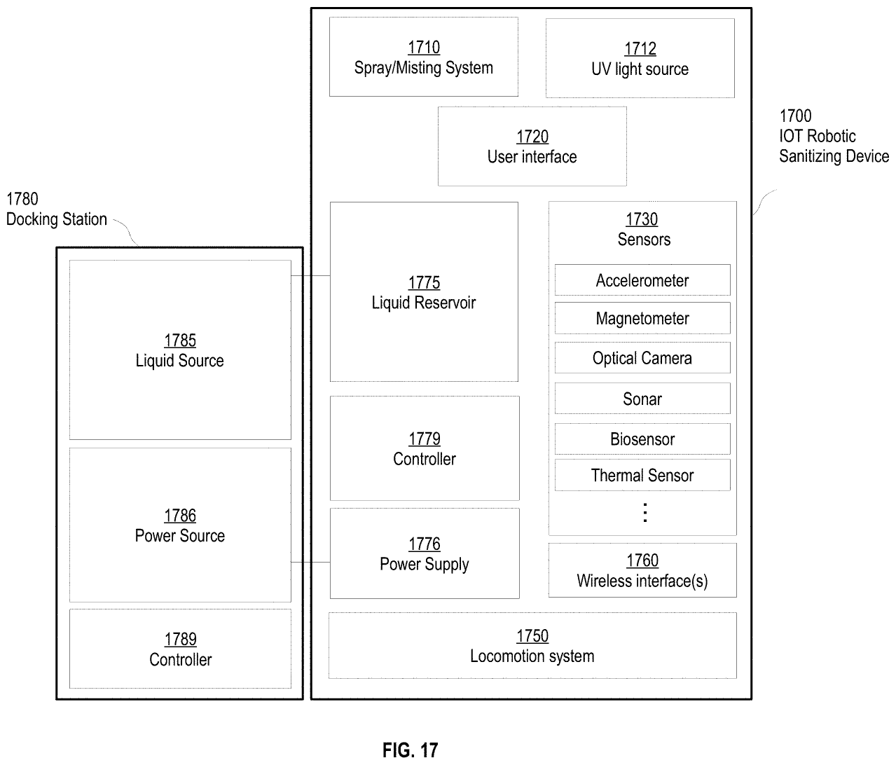

As will be described in greater detail below, the use of an IOT hub unit in conjunction with the sterilizing robot is particularly synergistic, since the IOT hub is the central in room or space controller (or other devices within the IOT network) can, for example, selectably controls and disables the HVAC system (to prevent premature venting of the disinfectant during the sterilization process) and remotely lock the door of the room (to prevent the entry of personnel during the sanitizing process). These and other aspects of the invention will be described in greater detail below in connection with FIGS. 17-26.

### Selected Definitions

As a preliminary matter, it will be understood that the term “sterilization” is used herein without loss of generality to refer to any process that reduces (either completely or in part) the level of infectious agents or pathogens (e.g., viruses, bacteria, fungi, mold spores, worms, and/or protozoa) present within the relevant environment That is, the terms “cleaning”, “disinfecting”, “sterilization”, and “sterilization” have similar but subtly different meanings in the art. “Cleaning” generally refers to the removal of germs, pathogen, dirt, and impurities from surfaces, but not necessarily the destruction of such pathogens. The term “disinfecting” generally refers to the use of chemicals, for example, EPA-registered disinfectants, to kill germs on surfaces. Disinfecting does not necessarily “clean” dirty surfaces or remove germs, but by killing germs on a surface after cleaning, it can further lower the risk of spreading infection.

The term “sterilization”, in some contexts, generally means reducing the level of pathogens on surfaces or objects to a safe level (using any combination of cleaning, disinfecting, etc.), often in accordance with an applicable public health standards or requirements. Finally, “sterilization” generally refers to a process that removes, kills, or deactivates all or nearly all forms of life, including pathogens.

As stated briefly above, then, “sterilization” is used herein without loss of generality to refer to any process that includes a form of disinfecting chemical agent that reduces (either completely or in part) the level of pathogens present within the relevant environment—whether or not that process meets the criteria of a particular public health standard. Additional information regarding these and other terms is available, for example, at the Centers for Disease Control and Prevention (see, e.g., https://www.cdc.gov/infectioncontrol/guidelines).

It should also be noted that, while the present invention is often described and illustrated using examples drawn from the hotel industry, the invention is not so limited. The sterilization systems described herein may be employed in the context of any interior space in which sterilization of the surfaces and environment is desired, such as hospitals, schools, churches, private residences, commercial office buildings, senior living, public spaces, apartment complexes, and the like.

The term “COVID-19” is used herein to refer to Coronavirus Disease 2019, known to be caused by the novel coronavirus SARS-CoV-2. Additional information regarding COVID-19 can be found, for example, at www.cdc.gov/coronavirus.

IOT Robotic Sterilization Device

Referring first to FIG. 17, a conceptual block diagram of an exemplary IOT robotic sterilization system will now be described. As shown, the system of FIG. 17 generally includes an OT robotic sterilization device (or simply “robotic sanitizer” or “robotic device” or “robot”) 1700 and a corresponding docking and charging station (or simply “docking station” or “dock”) 1780.

Robotic device 1700 generally includes a spray/misting system 1710, an ultra-violet (UV) light source 1712, a user interface 1720, a liquid reservoir 1775, a robot controller (or simply controller) 1779, a power supply 1776, a locomotion system 1750, a wireless interface or interfaces 1760, and a variety of sensors that might vary depending upon the application. The term misting is used without lack of generality to also include “clouding” and other aerosolizing processes. In the illustrated embodiment, for example, sensors 1730 include an accelerometer, magnetometer, optical camera, sonar, thermal (IR) sensor, and biosensor for airborne microorganisms. This list is not intended to be exhaustive, however, and might include a wide range of other sensors, detectors, and transducers—such as inertial measurement units (IMUs), gyroscopic sensors, acoustic sensors, proximity sensors, pressure sensors, force sensors, RFID readers, GPS modules, temperature sensors, and the like.

Docking station 1780, which is configured to electrically and mechanically interact with robotic device 1700, includes a liquid source 1785 (fluidically coupled, when docked, with liquid reservoir 1775), a power source 1786 (electrically coupled, when docked, with power supply 1776), and a docking controller (or simply controller) 1789, which may communicate either through a wired or wireless connection with robotic device 1700 and/or the IOT hub, which is described in further detail below.

Spray/misting system (or simply “misting system”) 1710 includes any combination of hardware (e.g., pumps, fluidics, nozzles, valves, tubing, etc.) configured to receive liquid from reservoir 1775 and convert that liquid to a cloud, fog, mist, spray, aerosol, or other form configured to treat the target surface and/or space. In one embodiment, for example, misting system 1710 is an electrostatic device. In such a system, the liquid source (from reservoir 1775) is combined with air and atomized by an electrode (driven by power supply 1776), and the resulting spray or cloud contains negatively charged particles that are able to aggressively adhere to all surfaces that are positively charged and allows it to coat surfaces evenly, and envelope objects-even if the sterilization robot does not have line-of-site to the target surface. After the spray is applied, the sterilization agent within the liquid works to sanitize the covered surfaces. In another embodiment, the system includes an ultrasonic transducer and fan combination that will produce an atomized aerosol mist from disinfectant dispensed from the onboard reservoir, which will be propelled around a space with the robot.

UV light source 1712 includes any source of UV light capable of assisting in sterilization surfaces and/or spaces within the environment. As is known in the art UV light (in particular, UV-C light) works primarily by destroying the DNA inside bacteria and viruses. In some embodiments, additional UV light sources controllable via the IOT hub are positioned within one or more ducts, air-conditioning units, or other components of the HVAC system to work in unison to sanitize air as it enters the room.

User interface 1720 includes any suitable combination of displays, user interface elements (e.g., buttons, dials, touch screens, LEDs etc.) provided to allow a human user to interact with the robotic device 1700. In many embodiments, robotic device 1700 is intended to be largely autonomous, and therefore user interface 1720 is designed to be provide only minimal functionality.

Liquid reservoir (or simply reservoir) 1775 is preferably configured to hold enough liquid to complete a full sanitization session without replenishing its supply (e.g., via liquid source 1785 of docking station 1780). A wide range of disinfecting liquids may be used in connection with reservoir 1775 and misting system 1710.

In one embodiment, hypochlorous acid (HOCl) is used as the disinfecting fluid. HOCl is a naturally occurring substance that is recognized by the Environmental Protection Agency (EPA) to be Effective Against SARS-CoV-2 (the virus that causes COVID-19) as a very effective disinfectant in the chlorine family, due in part to its ability to penetrate the cell walls of viruses and bacteria.

Wireless interface 1760 may include any combination of hardware for communicating with the IOT hub and other components in the environment and IOT network. Nonlimiting examples include Wifi, Zigbee, Zwave, Bluetooth, and LoRa (or LoRaWAN); however, any wireless communication methods now known or later developed may be employed.

Locomotion system 1750 includes any combination of hardware, control systems, and other electro-mechanical components configured to allow movement of robotic device 1700 along a prescribed or autonomously determined path through its environment. In some embodiments, for example, locomotion system 1750 includes one or more drive wheels configured to allow movement (i.e., circular, linear, two degrees of freedom) such that it can navigate the unobstructed areas of a floor within the room. The invention is not so limited, however, and may include any suitable locomotion system for two-dimensional and/or three-dimensional navigation through a space.

Controller 1779 includes any combination of hardware, software, and/or firmware configured to communicate with the other subsystems and modules of FIG. 17, and to carry out—alone or in combination with other external processing systems—the functionality described herein. Controller 1779 also preferably includes any software required for determining a path to take during a sterilization session (which may be previously determined or determined autonomously using an appropriate machine learning algorithm).

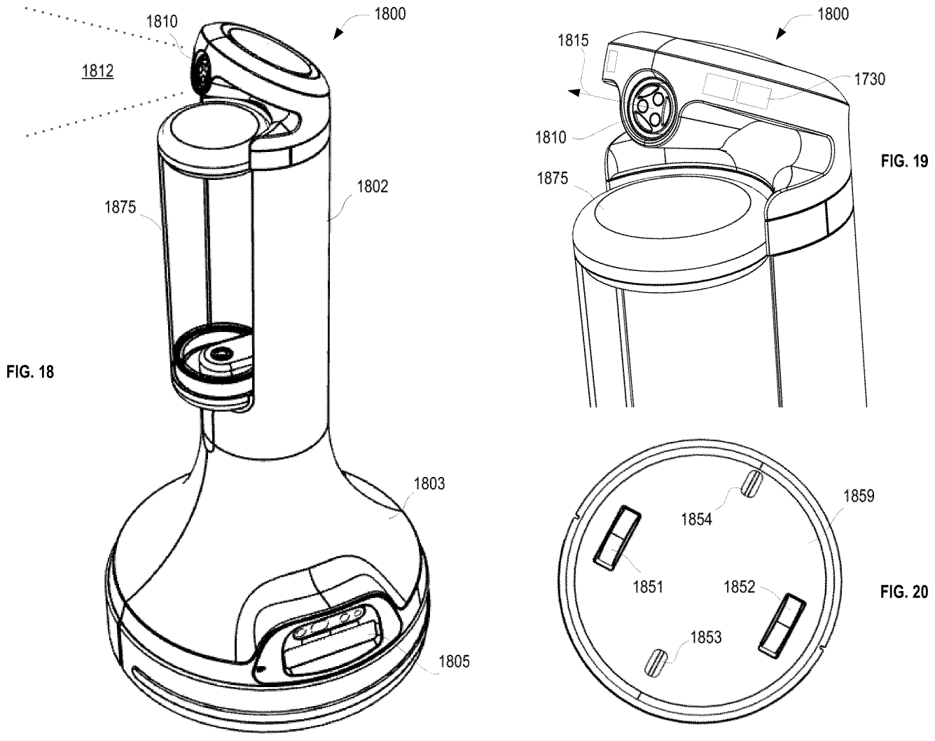

It will be appreciated that the system illustrated in FIG. 17 may be implemented in a variety of ways and may be designed to exhibit a wide range of shapes and sizes. FIGS. 18, 19, and 20, for example, are isometric, close-up, and bottom views, respectively, of an example IOT robotic sterilization device in accordance with one embodiment. That is, referring to FIG. 18, robotic device 1800 has a base portion 1803 and a vertically extending body portion 1802. User interface components 1805 (corresponding to 1720 in FIG. 17) are provided within the base portion 180, while the misting system 1810 is provided at a minimum near the top of body portion 1802 (as shown in FIG. 19), collectively referred to as the “tower.” Multiple dispensing angles and levels of deployment may be provided—e.g., 360 degrees near the top, single direction, bi-directional, and/or mid-tower.

A reservoir either replaceable or permanent 1875 is provided within body portion 1802 and is fluidically coupled to misting system 1810 to produce a mist, spray, cloud, or vapor pattern 1812 which, in this embodiment, has a directional component defined by the axis of the nozzle or other components of misting system 1810 (i.e., axis 1815 illustrated in FIG. 19). Axis height from floor 1815 may be oriented in a variety of ways, such as horizontal (parallel to the ground plane), straight up (to produce a vertical cloud), or at an intermediate angle (e.g., 30-60 degrees from horizontal). In some embodiments, misting system 1810 can telescope or articulate relative to body 1802 to achieve a variety of spray patterns. In some embodiments, the reservoir is internal and cannot be seen from the exterior of the body, thus maintaining a sealed and tamper resistant enclosure that can only be accessed by a certified technician or authorized facility worker or be self-filled through a separate reservoir station that the unit will navigate to for charging and refilling to reduce human labor to constantly till the reservoir leading to continuous autonomous sterilization operation over a period of time or for larger spaces.

In one embodiment, the height of misting system 1810 relative to the ground (i.e., the bottom of base 1803), ranges from 3 to 6 feet, which may vary depending upon application, and is not intended to be limiting.

In the illustrated embodiment, as shown in FIG. 20, the bottom of the unit 1859 includes a pair of independent drive wheels 1851, 1852 and a pair of stabilizing passive wheels 1853, 1854, or three to four omni-directional wheels or Mecanum wheels. Through proper control of wheels 1851 and 1852, robotic device 1800 can be made to rotate and translate to an arbitrary or predetermined location in a planar region.

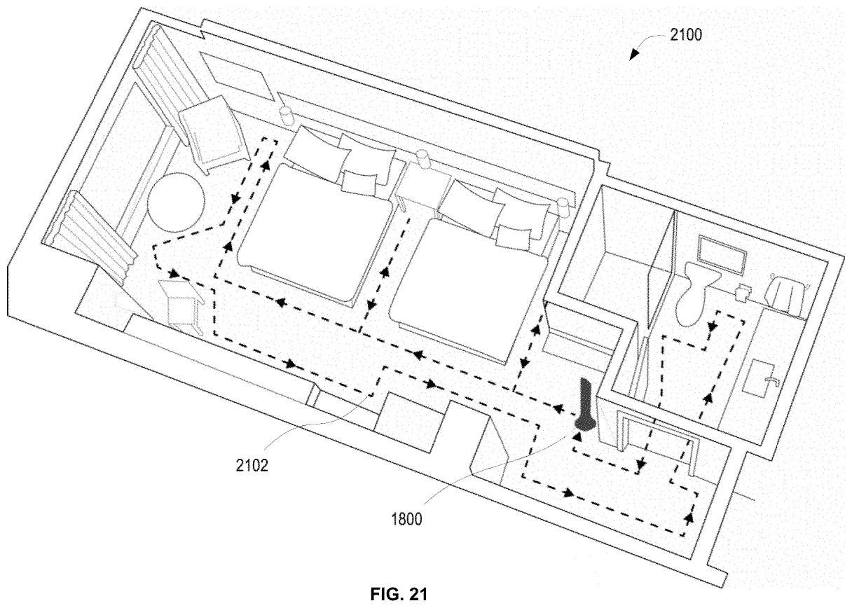

FIG. 21 is a cut-away, isometric view of an example room showing the example path that a robotic sterilization device may take during a sterilization session. That is, as one non-limiting example, a room 2100 is illustrated with a number of rooms, walls, doorways, and furniture as shown. This pattern or method is not limited to a hotel room but can be applied to any type of room or space such as an office, healthcare facility, classroom, office and more that are utilized by people during the course of a day or night. The robotic device 1800 begins at a particular location (e g., a docking/charging/filling station within room 2100) or enters from outside the room, then follows the path indicated by dotted line 2102 until it ultimately reaches its starting point. The path 2102, as mentioned previously, may be pre-programmed or learned adaptively during the course of a sterilization session. For example, in the hotel room scenario, the guests may have left a suitcase or other object along the expected path 2101. In such case, the robotic device 1800 may determine that an area cannot be accessed, making note of that deficiency of sterilization and communicate that relative portion of room 2100 was not properly sterilized via an mobile app or backend portal that will alert the housekeeping or janitorial staff. It will be appreciated that, in addition to the path 2102, the robotic device 1800 will also generally rotate to a particular orientation (or rotate through a particular arc) to provide adequate coverage. The angular motion of robotic device 1800 may also be predetermined (i.e., “hard-wired”) or determined adaptively during a sterilization session. In the case of obstruction to the predetermined sterilization path, the robotic device will attempt to circumvent the obstruction so that it can complete the sterilization routine.

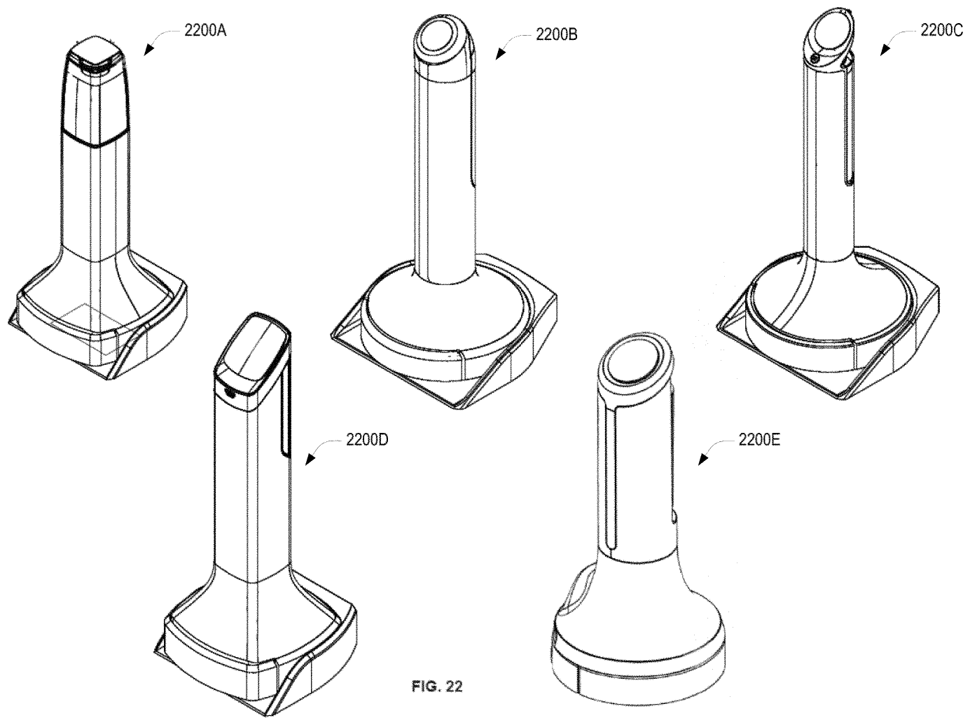

FIG. 22 illustrates various alternate designs (2200A, 2200B, 2200C, 2200D, and 2200E) for a robotic sterilization device in accordance with the present invention, with associated base docking units (associated liquid source not shown). As can be seen, these designs show a variety of reservoir locations and misting angles. Device 2200A, for example, shows a misting axis oriented substantially vertically, as discussed briefly above.

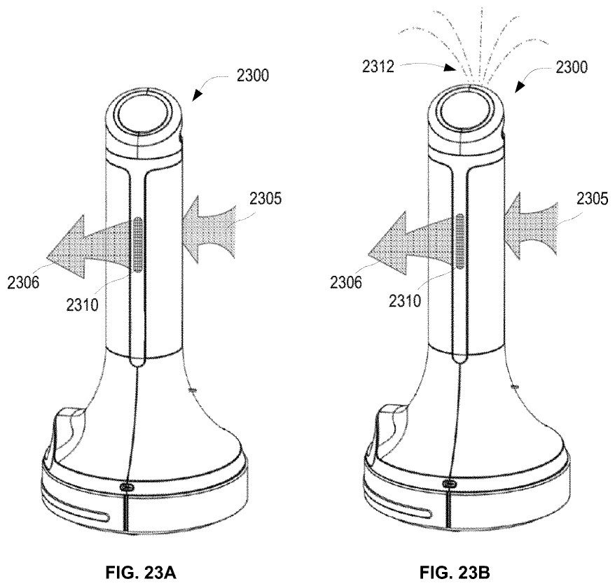

FIGS. 23A and 23B illustrate the use of an internal biosensor that can constantly monitor for airborne pathogens to determine when the robotic sterilization device should begin and end the sterilization process. That is, the body of robotic device 2300 includes an air inlet and outlet (2310) through which a sample of room air can be drawn and expelled (2305, 2306), and an internal biosensor configured to sense the presence of pathogens in that sample. In the event that the level of pathogens in the sample rises above a predetermined threshold (which may vary depending upon the nature of the pathogen), the robotic device 2300 begins the sterilization process (illustrated as spray 2312 in FIG. 23B) If the level of pathogens falls below the predetermined threshold, then robotic device 2300 terminates the sterilization process.

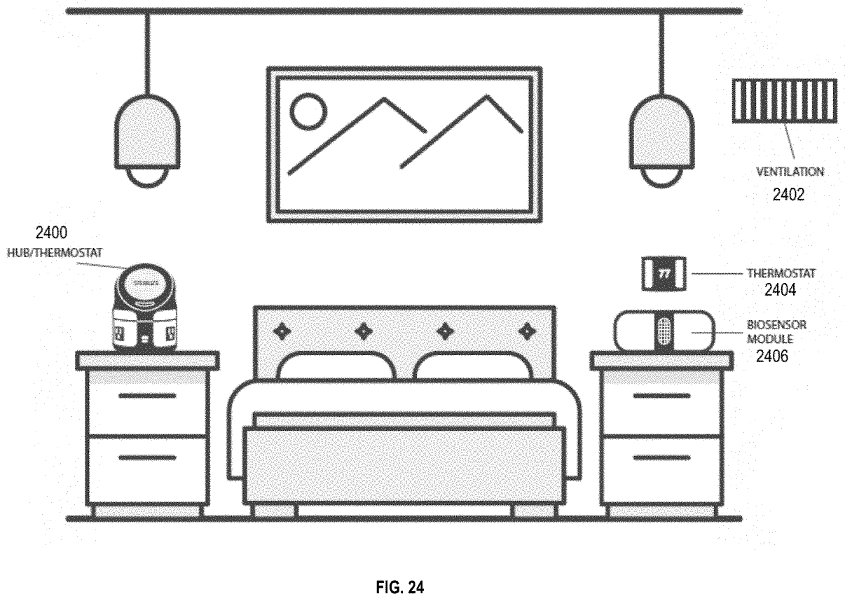

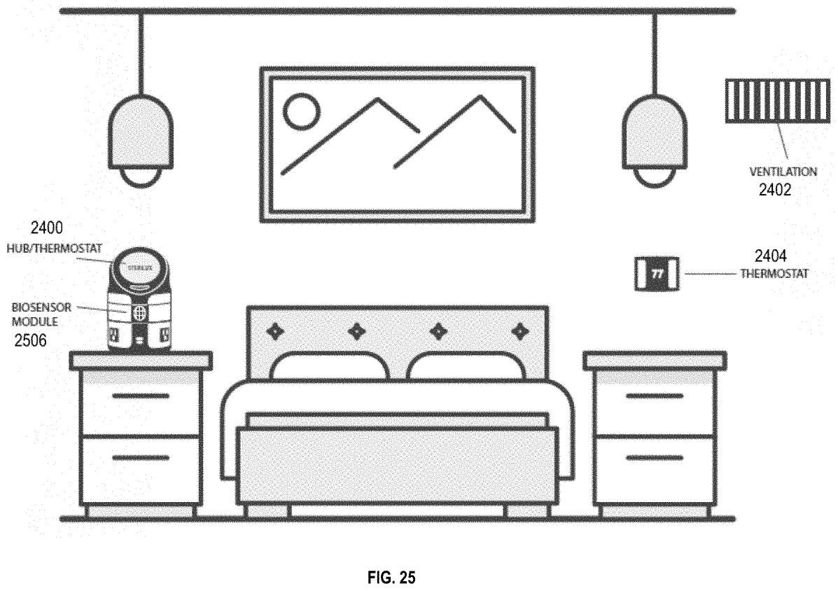

It will be appreciated that the biosensor module provided within robotic device 2300 in FIG. 23A may also be incorporated into the environment itself. FIG. 24, for example, illustrates the interior of a room with associated bedstand biosensor IoT module appliance 2406, ventilation system 2402, thermostat 2404, and IOT hub/thermostat 2400. In this embodiment, hub 2400 can control biosensor IOT module appliance 2406 and thermostat 2404 to turn off air flow and begin sampling the air for pathogens. If the pathogen level is found to be above a predetermined threshold, then the robotic device (not shown in FIG. 24) can be activated —then later returned to its dock when the sterilization process is complete FIG. 25 illustrates a variation of the room interior shown in FIG. 24, in which the biosensor module 2506 is integrated into the IOT hub to detect pathogen level in the air. This consolidates devices into one and reduces the footprint on a table. The Hub will then be able to turn on the HVAC and circulate in fresh air to further lower the particle density of the pathogens in the air. Furthermore, IoT UV lights may be located in the discharge of a variety of HVAC vents to further eliminate or reduce the aerosolized pathogens.

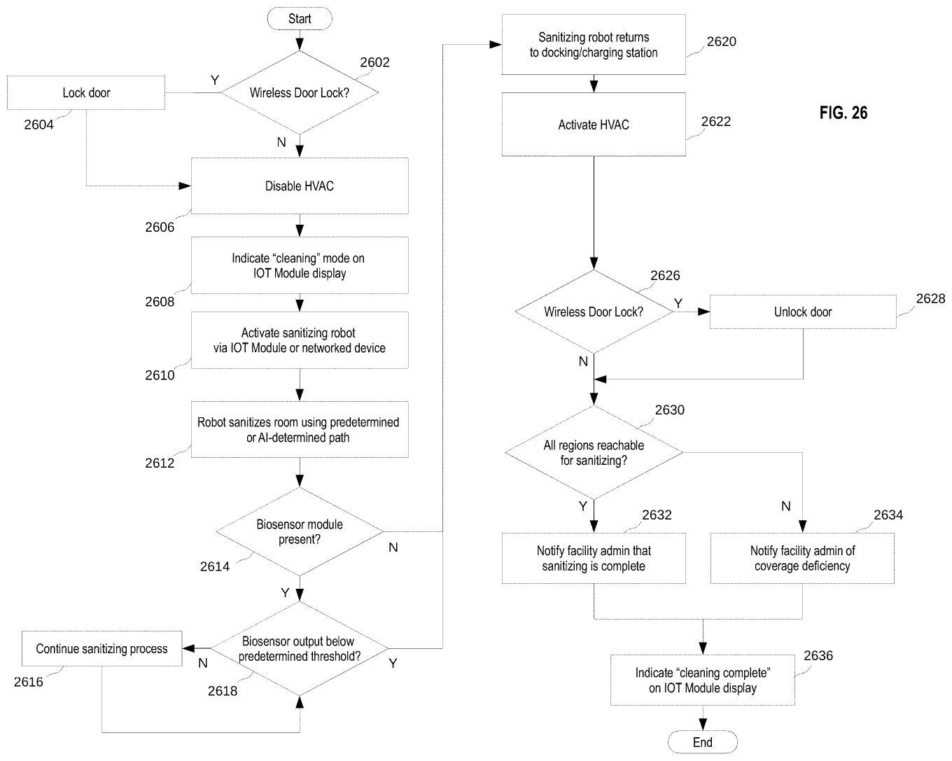

FIG. 26 is a flowchart depicting a sterilization method in accordance with an exemplary embodiment. Depending on the liquid or chemical used to sterilize the space through the device the room may or may not be locked to avoid people from entering a space until the sterilization process and contact time is complete. If the liquid is safe for people then the step of locking the door may not be necessary. The process itself may be initialized in a number of ways and by a variety of entities, such as through a Property Management or Building Management system, preprogrammed events such as in-between school class sessions, company meetings, guest check-out, or dinner seating reservation time, the robotic device itself, the IOT hub (in conjunction with biosensor module 2406), or a remote user making a request via networked computing device.

Initially, at 2602, the system determines through occupancy sensors whether anyone is present in the room at which time the Hub will activate the door lock to the room or other space in a controllable manner (e.g. whether the door lock itself is an IOT device). If so, the door is locked (2604) (to prevent the entrance of guests or other individuals), if not, the process continues to step 2606, whereupon the HVAC system for the room is disabled. That is, the air handler is prevented from altering the spray pattern or coverage or by further dispersing the disinfecting mist produced by the robotic device during the process and inhibiting the sterilization process ineffective thus exposing workers and people to contaminated pathogens raising the infection potential.

Next, at 2608, the IOT hub display is changed to reflect that the system has entered a sterilization mode (e g., via lighting, a text or graphic display, etc.), and the IOT sterilization robot is activated to begin the sterilization process (step 2610). The sterilization robot then follows a path through the room while producing the disinfecting mist (step 2612) The path may be predetermined (i.e., from a previous session), or may be determined adaptively during the session itself.

Next, at 2614, a determination is made as to whether a biosensor module is present (e.g., within the robotic device itself, or as a separate module provided within the room). If so, then sterilization continues while the sensed pathogens in the room are above some predetermined threshold (conditional loop 2618, 2616); if not (and/or when the pathogen level false below the predetermined threshold), then the system continues to step 2620, and the robotic device returns to its docking/charging station.

At this point, the HVAC system can be activated to circulate in fresh air to reduce the particle count of pathogens in the air as well as remove any excess disinfectant in the air (step 2622), and any controllable door locks are unlocked (steps 2626 and 2628).

Next, the system determines whether all regions of the room were reachable for sterilization (step 2630). That is, as mentioned previously, it is often the case that an object may have been placed in the intended sterilization path prior to activation. In such cases, the robotic device stores that information—e.g., the points at which the path diverged from the intended, optimal path. If all regions were reachable, then processing continues with step 2632, and the facility administrator or worker, housekeeping, janitorial, guest or teacher (or other party) is notified that sterilization is complete. Otherwise, the facility administrator or worker is notified of a coverage deficiency (step 2634), which may prompt an additional sterilization step at a subsequent time, for example, after any obstacles have been removed from the path. This follow-up sterilization step might be configured to treat only the areas not previously covered. The IOT hub user interface is then modified to reflect that cleaning is complete (step 2636) and the room sterilization is communicated through a mobile app to hotel guests showcasing the multiple step process, date time completed to give additional confidence that the space was sterilized.

In accordance with one embodiment, an IOT “scent-dispensing” appliance can be included within the interior space. Such a device is configured to dispense a burst of scent to present a fresh aroma after the sterilization process. In the hotel context, a guest may select his or own preferred scent, which can be stored in a profile for that guest.

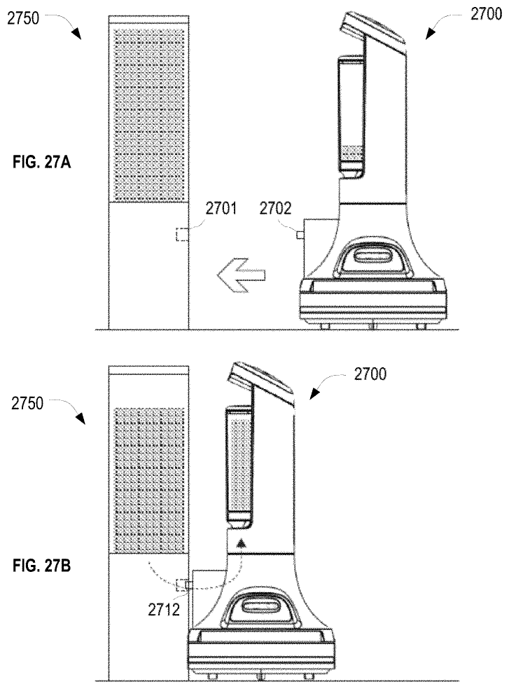

FIGS. 27A and 27B illustrate the use of a docking/refill station 2750 in connection with a robot 2700. As shown in FIG. 27A, refill station 2750 includes a port 2701 configured to fluidically couple, temporarily, with a corresponding coupling 2702 provided on robot 2700. In this way, the system can replenish the reservoir of robot 2700 using a supply of liquid provided by refill station 2750 (arrow 2712 in FIG. 27B), which might be manually refillable or plumbed to a liquid source provided in the room. While not illustrated in the figures, it will be understood that refill station 2750 and/or robot 2700 will include suitable pump and other fluidic components necessary to effect transfer of the liquid or the robot may have the capability with an onboard pump to fill its own reservoir which docked to the refill station that may or may not have charging capabilities.

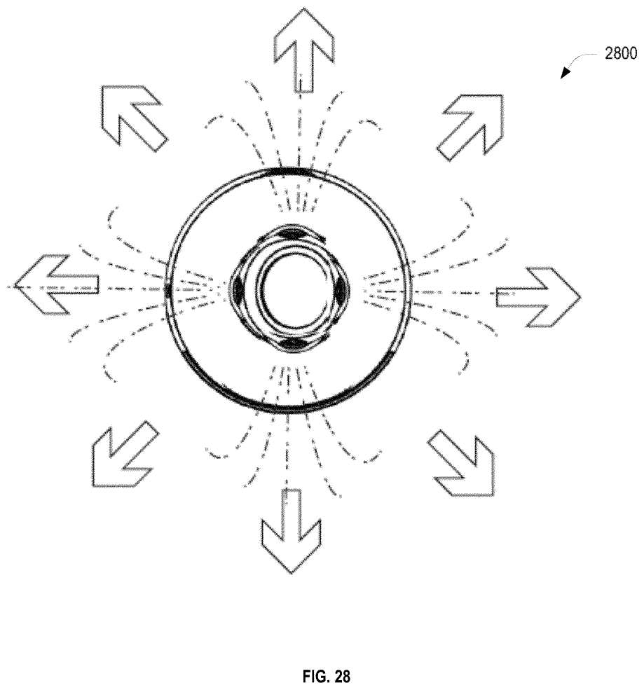

The spray/dispensing system of the present invention may be configured to spray, mist, or fog a region in accordance with a wide range of spray patterns, such as uni-directional, bi-directional, or omnidirectional. For example, FIGS. 28, 29, and 30 illustrate example spray patterns in accordance with a number of non-limiting embodiments. FIG. 28 is a top view 2800 of a robotic device in which the mist is dispensed from four ports oriented at 90-degree angles relative to each other That is, the mist is dispensed substantially uniformly (omnidirectionally) in the plane. While FIG. 28 illustrates four ports, any number of ports (e.g., 5 or greater ports) may be used to achieve a substantially uniform pattern as shown. Additionally, the arrow shows that the robot through use of lidar, radar or other navigational technology can traverse a space in an infinite number of directions providing circumferential sterilization mist for fewer path trips within a given space.

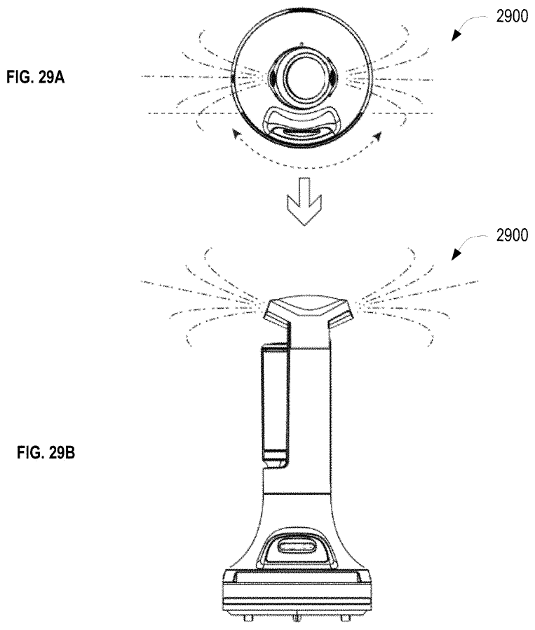

FIGS. 29A and 29B illustrate, respectively, top and side views of a robot in which the spray pattern 2900 is bi-directional and perpendicular to a navigational and detection sensors for the robot and perpendicular to the direction of travel to optimize the sterilization of objects within the space thus keeping the navigation sensors from being occluded by the mist or fog. That is, two antipodal ports (situated at a 180-degree angle relative to each other) are provided to dispense the mist on opposite sides of the robot as shown. The robot may be rotated to control the region or regions covered by the mist during movement.

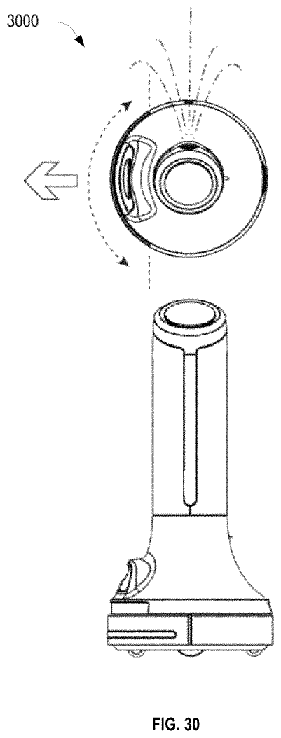

FIG. 30 illustrates another embodiment in which a single port is used to create a uni-directional spray pattern 3000, which in some embodiments is perpendicular to a navigational and detection sensors for the robot and perpendicular to the direction of travel to optimize the sterilization of objects within the space thus keeping the navigation sensors from being occluded by the mist or fog. In this example, the port is oriented at 90 degrees (perpendicular) to the direction of motion of the robot itself; however, the invention is not so limited, and may be oriented at any suitable angle relative to the robot body.

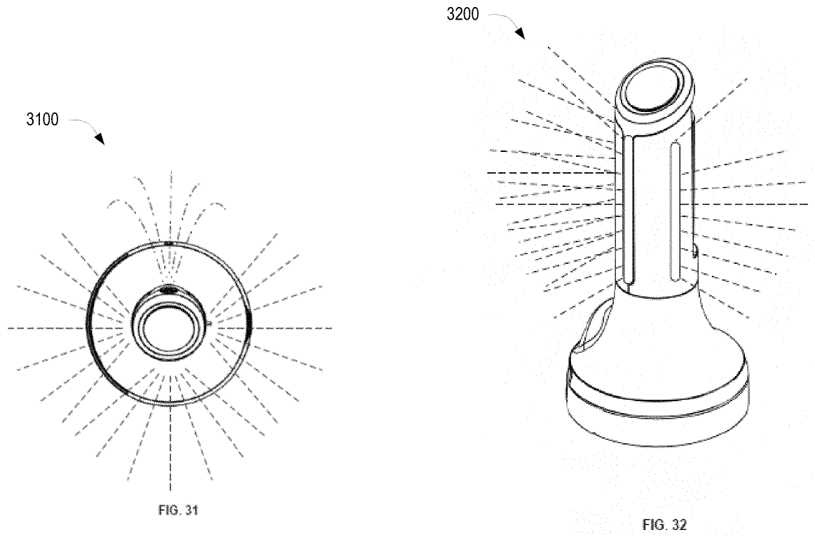

FIGS. 31 and 32 illustrate, respectively, top and three-quarter views of a robot (3100, 3200) characterized by dual functionality including a misting feature in combination with at least one UV Light integrated into the body of the device to irradiate the immediate space being treated for added efficacy in eliminating pathogens.

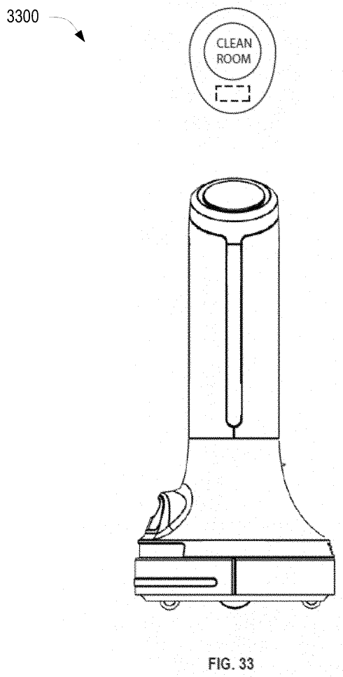

FIG. 33 illustrates a wall-mounted IOT Hub (3300) that includes a display to communicate the room sterilization process along with integrated occupancy sensors (dotted rectangle) configured to determine if any humans are present in the room being sterilized by the IoT device.

Any of the above systems, such as path determination, sterilization schedule, etc. may be implemented using one or more machine learning models. The phrase “machine learning” model is used without loss of generality to refer to any result of an analysis method that is designed to produce some form of prediction, such as predicting the state of a response variable, clustering variables, determining association rules, enabling robotic swarming capabilities to further sterilize or treat areas with other chemical agents and performing anomaly detection. Thus, for example, the term “machine learning” refers to models that undergo supervised, unsupervised, semi-supervised, and/or reinforcement learning. Such models may perform classification (e.g., binary or multiclass classification), regression, clustering, swarming, dimensionality reduction, and/or such tasks. Examples of such models include, without limitation, deep learning models, artificial neural networks (ANN) (such as a recurrent neural networks (RNN) and convolutional neural network (CNN)), decision tree models (such as classification and regression trees (CART)), ensemble learning models (such as boosting, bootstrapped aggregation, gradient boosting machines, and random forests), Bayesian network models (e.g., naive Bayes), principal component analysis (PCA), support vector machines (SVM), clustering models (such as K-nearest-neighbor, K-means, expectation maximization, hierarchical clustering, etc.), and linear discriminant analysis models.

It will be appreciated that, while the IOT robotic control methods described herein are not limited to a sterilization system that dispenses liquid disinfectant, and may be used in connection with other forms of liquids in other contexts. For example, in a hotel, office or school context, the system may be adapted to enabling robotic swarming capabilities to further dispense aqueous pepper-spray or some other deterrent in the vicinity of an active shooter or other intruder that has gained access to a facility ahead of first responders arriving to impede, incapacitate or debilitate the progress of an active shooter. In the event that wireless locks are available via the IOT network, the system may also lock doors as appropriate to limit the movement of the intruder. Furthermore an in-room hub will have integrated or IoT Edge occupancy sensors that will detect human presence in a space and signify their presence in a backend interface or mobile app to rapidly and immediately assist first responders during their search and rescue efforts to save lives and eliminate the threat. Furthermore, multiple such IOT robotic devices may team up and “swarm” an intruder. The location of the intruder may be determined by the robotic devices themselves with integrated onboard shot detection microphones or sensors or via another networked IoT microphone sensor components and/or machine learning system with access to surveillance images of classrooms and hallways.

IOT Base Module and Network

Having thus provided a detailed description of an IOT sterilization device in accordance with various embodiments, an exemplary IOT base module and associated IOT network will now be described.

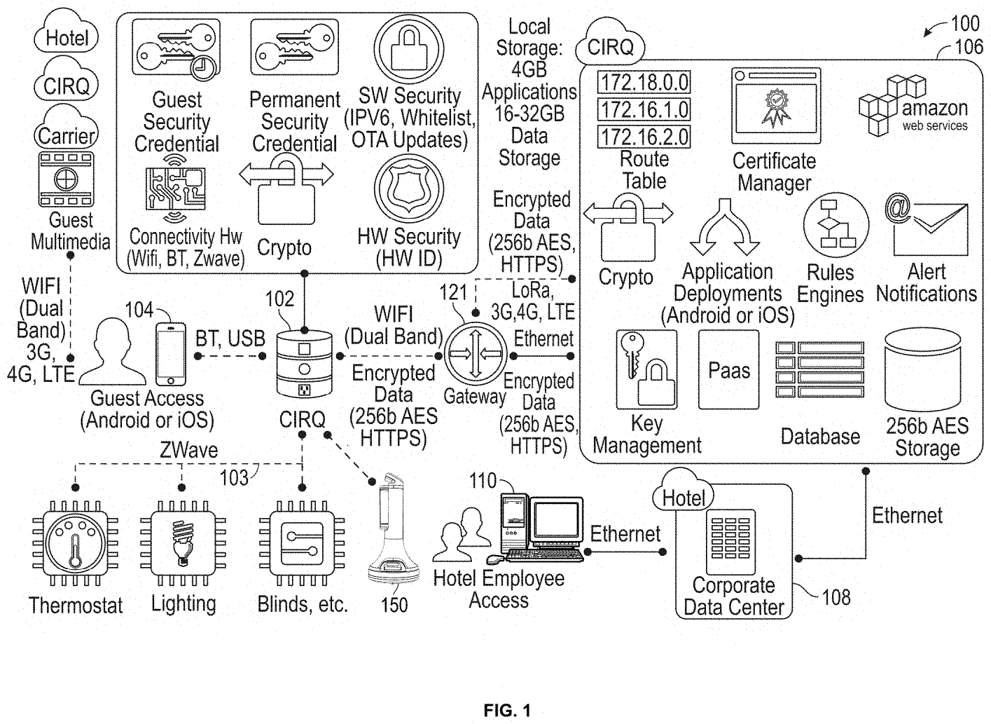

In that regard—referring now to FIGS. 1-16—presently known mobile apps for controlling IOT devices offer a limited value proposition to the user, and are typically limited to consolidating multiple end point assets (e.g., lighting, door locks, HVAC) into a connected central guest room hub. In contrast, the present system offers a more robust value proposition to the guest user in the form of enhanced control of the user experience, amenity upgrades, rewards, personalization preferences and targeted/push marketing messages, offers and notices as well as enabling features (both on and off property) that would otherwise be unavailable without location services enabled.

Various embodiments contemplate monitoring the guest's location using the guest's mobile phone, wearable accessory, laptop, or any other GPS or location-enabled device. In this way, location aware (and hence context aware) features and services may be pushed to the guest in new and imaginative ways heretofore not contemplated by existing systems. Moreover, by incenting the guest to keep location services enabled even when the guest is off the hotel property, valuable tracking information may be collected, mined, and harvested to design precisely designed marketing messages delivered with pinpoint accuracy. An additional benefit of collecting aggregate location data surrounds the ability to conduct advanced analytics, and to offer customized guest benefits with guest room and property wide preferences based on these analytics.

By way of non-limiting example, contextual awareness may include “knowing” that an individual is primarily or currently attending to business or pleasure, the guest's short term and/or long term itinerary, the guest's previous locations (conference room, restaurant, office building, movie theater), and unique user preferences relating to cuisine, entertainment, lifestyle, music, and environmental comfort metrics such as lighting, room temperature, mattress firmness, and the like. Additional contextual awareness metrics may include monitoring when location data goes dark, and thereafter re-emerges in another city, suggesting that the guest has flown from one city to another.

In other embodiments the resort (e.g., front desk) can use the control modules to broadcast messages (e.g., active shooter or other customized messages, notifications, alerts, instructions, warnings, or other emergencies or priority notices) to all rooms on or off the premises, a subset of rooms based on guest profiles or demographics (e.g., all convention participants), a particular wing or building, or the like.

Recognizing that even when a device is “off,” it may still be listening for an “on” or “listen” command, the control module may include a manually slidable, disengageable, or otherwise configurable button or mechanical feature to physically disconnect the voice processor, microphones, sensors and other associated components thereby electrically unplugging and deenergizing the voice capture hardware to ensure guest privacy and data security.

Other embodiments contemplate employee wearable panic button modules which may be Bluetooth or WIFI connected to beacons positioned within the hotel property or used through the employee's mobile phone. The system may be configured to track employees and to alert emergency personnel if an employee is assaulted or otherwise in need of assistance while on property. Accelerometers may be incorporated into the panic button module to detect a fall, and record audio if triggered. The panic signal emitted by the panic module can be detected by beacons located throughout the property, and a geo-fence violation broadcast when someone leaves the property. One embodiment, the panic button communicates with the CIRQ device; in other embodiments the panic buttons communicate with beacons located around the property.

Occupancy sensors and/or voice recognition systems may be employed keep track of all people in the room, permitting the control module to engage in multi-party conversations, or plural single party exchanges.

Other embodiments contemplate fragrance pods (e.g., tied to a cloud based control system) located in guest quarters, meeting rooms, and other guest areas. The pods may be configured to dispense predetermined fragrances selected by the hotel or the guest. The scent can also be subliminal, to enhance the mood and personification of the display screen, e.g., using aromatherapy, essential oils, popular food items, ocean breeze, rain forest, and the like.

Additionally, the PaaS system may be configured to learn individual habits, routines and preferences to intelligently (e.g., algorithmically) prepare the hotel room to accommodate desired environmental factors including temperature, lighting, window shade position, entertainment, and consumable items such as beverages and food that would deliver an inviting and welcoming room presence to drive loyalty for the property and brand.

From an enterprise standpoint, the system contemplates at least the following levels of value proposition: i) allowing the property to offer guests the ability to control and manage a plurality of IOT devices in the room using a mobile app, with low hardware and installation costs; ii) wirelessly controlling room temperature through a controller mounted within a bedside module; iii) thermal mapping and motion mapping using multiple sensors within a guest room to monitor occupancy through presence or respiration; iv) promoting conservation through gamification coupled with a loyalty rewards component; v) providing the guest with perks and other features which leverage location services (tracking); vi) mining the resulting aggregate location data facilitates the development of enhanced targeted marketing programs; and vii) allowing the hotel property the ability to substantially reduce power and water usage within each room. Viii) the ability to migrate personal environmental and personal preferences from property to property.

The value proposition to the guest includes providing enhanced information to the mobile device thru the mobile app regarding the environment within and outside the room on their personal devices or in another functional use having the app running on the central hub. In various embodiments, this involves a cloud based system server (sometimes referred to herein as the CIRQ server) operating within the broader internet environment to thereby integrate the immediate environment (guest room) with the extended environment (the resort property, nearby attractions, and remote attractions).

In various embodiments, the in-room IOT control module is used to drive initial user engagement including operating a version of the mobile app and enabling guest connectivity and services, whereupon the resulting location awareness (tracking) may be used to drive further user engagement (e.g., on and off property perks, targeted and push marketing). Aggregate tracking data from multiple users may then be mined and harnessed to drive further targeted marketing notices, offers, messages, schemes, energy savings, and to analyze travel and spending trends. Indeed, the intersection among the PaaS System with in-room IOT control and location awareness alone has significant value in terms of energy savings for the property owner, as described below.

In addition, the system may be configured to gather performance data for the IOT devices and appliances, failure modes and trends, lifetime usage, servicing cycles/predictions and duty cycles in multiple geographic locations to thereby reduce long term total cost of use, increasing revenue/profit for the PaaS System and driving capital equipment replacement and upgrade timetables for property owners.

Turning now to FIG. 1, a system 100 for providing enhanced customization to a guest experience includes an in-room IOT module 102 (or “IOT base module”) for controlling a local IOT network 103 (e.g., robotic device 150), an associated mobile app running on a guest mobile device 104, an enterprise server 106 including a PaaS platform, and a property owner server 108 configured to communicate with a facilities controller 110.

More particularly, the IOT module 102 is configured to communicate with the guest device 104 using Wi-Fi, Bluetooth, wired or wireless Ethernet, VPN, USB, Zigbee, Z-Wave, cellular (3G, 4G), or any radio bands other suitable wired or wireless protocol. The IOT module 102 is configured to communicate with the devices which comprise the IOT network 103 using ZWave, Bluetooth, or any suitable wired or wireless protocol. The IOT module 102 is configured to communicate with the enterprise server 106 through a gateway 121 (such as the internet) using Wi-Fi, LoRa, 3G, 4G, LTE, Ethernet, radio or any suitable wired or wireless protocol. Similarly, the enterprise server 106 is configured to communicate with the property owner server 108 using Wi-Fi, LoRa, 3G, 4G, LTE, Ethernet, radio or any suitable wired or wireless protocol.

In a typical use case, the guest device 104 communicates directly with the IOT module 102 when the guest device 104 is inside or otherwise closely proximate the hotel room. When the guest is outside the hotel room, off the hotel premises, or otherwise out of range of the IOT module 102, the guest device 104 communicates directly with the enterprise server 106 using a cellular network (e.g., 3G, 4G, LTE) radio or through a suitable wired or wireless internet connection.

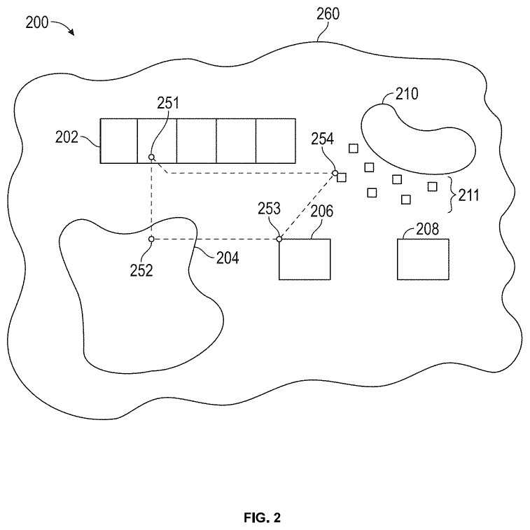

FIG. 2 is a schematic diagram of a hotel property 200 illustrating tracking data for a hotel guest within the boundary of the hotel property. In the illustrated example, the mobile app tracks the guest's movement from a guest room 202 (point 251), to golf course 204 (point 252), to a restaurant 206 (point 253), to a particular one of a plurality of cabanas 211 adjacent another structure 208 and a pool 210 (point 254), and back to the room (point 251). A geo-fence 260 defines the boundary of the hotel, resort, or time share property.

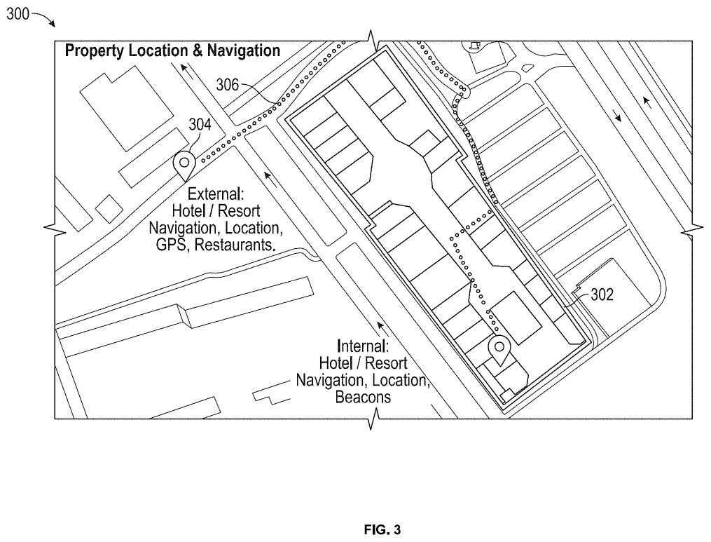

FIG. 3 is a schematic diagram 300 illustrating tracking data 306 for a hotel guest within and outside the boundary 302 of a hotel property as the guest visits an off premises location 304 (tourist attraction, restaurant, office, theater, or the like).

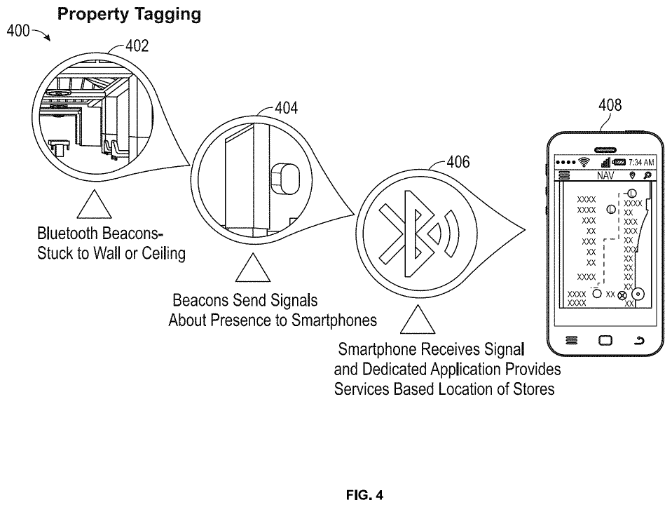

FIG. 4 is a schematic diagram 400 illustrating the use of beacons in addition to and/or in lieu of traditional GPS based location services. More particularly, FIG. 4 depicts a hallway 402 including wall mounted beacons or embedded building or infrastructure sensors. Each beacon 404 is configured to send static location information to the mobile app using Bluetooth or similar protocols 406. In this way, even without enabling location services, the mobile app can display the locations of various hotel amenities on the mobile device screen 408.

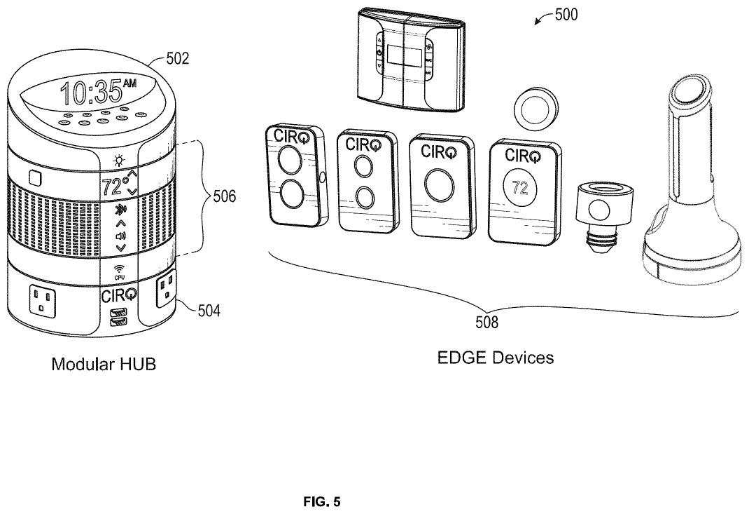

Referring now to FIGS. 1 and 5, an exemplary PaaS system with an in-room IOT network system controller 500 includes a control module 502 and a plurality of IOT devices (referred to herein as Edge devices) 508. In particular, the control module 502 includes a base 504 and a plurality of stacked electronic modules 506, each of which is configured to communicate with, monitor, and/or control one or more of the edge devices.

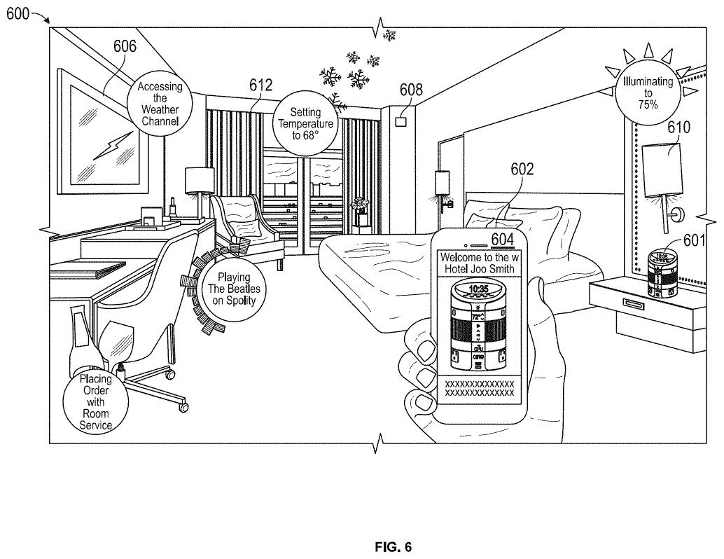

FIG. 6 is a schematic diagram 600 illustrating a mobile device 602 operating a mobile app 604 for controlling an IOT module 601 which, in turn, coordinates a plurality of IOT devices such as, for example, an entertainment system (e.g., television) 606, a thermostat or other HVAC controller 608, lighting 610, motorized window coverings 612, and a services module (not shown) for coordinating resort amenities (e.g., room service, reservations for local restaurants and tourist attractions).

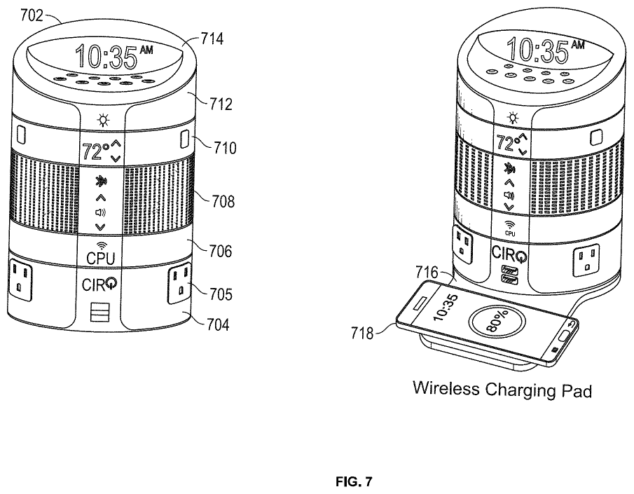

FIG. 7 is a more detailed view of an exemplary IOT control module 702 including a base 704 having one or more female AC adapters 705 (illustrated at left), and a CPU module 706 including a Wi-Fi component, a ZigBee Multi-Band IoT Mesh Network Technology component, and/or a hard drive component. The control module 702 further includes an audio module 708 including a speaker and/or microphone component, a sensor module 710 including a remote thermostat module with thermo-sensors and ultrasonic sensors and motion and/or infrared sensor module, a smart LED module 712, and a utility module 714 including a digital alarm clock, a radio, and an optional mobile docking/charging station. An alternative embodiment (illustrated at right) of a mobile docking/charging station 716 is shown charging a smart phone 718. In this regard, the sensor module 710 may also include one or more radar antennas in the head unit configured to triangulate with the relay radar antenna to facilitate occupancy detection.

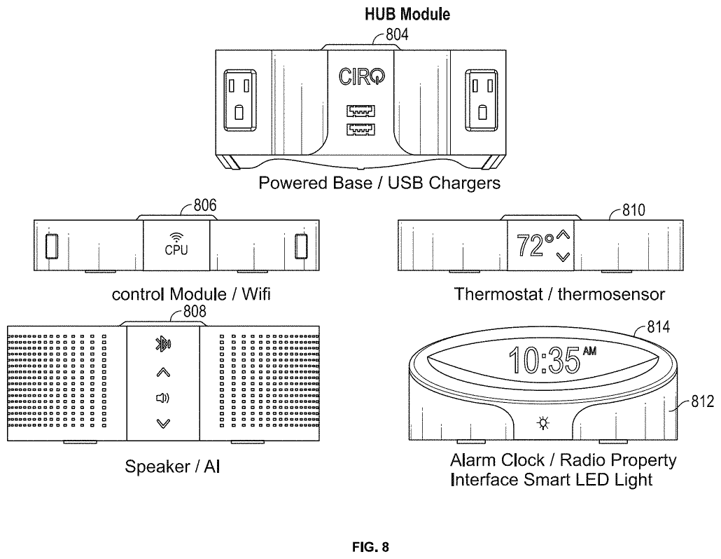

FIG. 8 is a schematic view of the stackable electronic hub modules shown in FIG. 7, including a base module 804, a CPU module 806, an audio module 808, a remote thermostat and occupancy/thermo-sensor module 810, a lighting module 812, and a utility module 814.

FIG. 9 is a schematic view of an IOT controller 902 disposed between a first bed 904 and a second bed 906 in a typical hotel, resort, or time share room environment. In the illustrated embodiment, the modular stack may include a remote relay to be used in the place of a traditional wall thermostat with the motion, radar, and/or infrared sensors (not shown) may be positioned so that full room coverage may be obtained using a minimum number of sensors (e.g., 2). Additionally, by having the remote thermostat bedside the guest will be able to adjust the temperature controls on the remote thermostat and user interface and as well using the mobile app without leaving the bed.

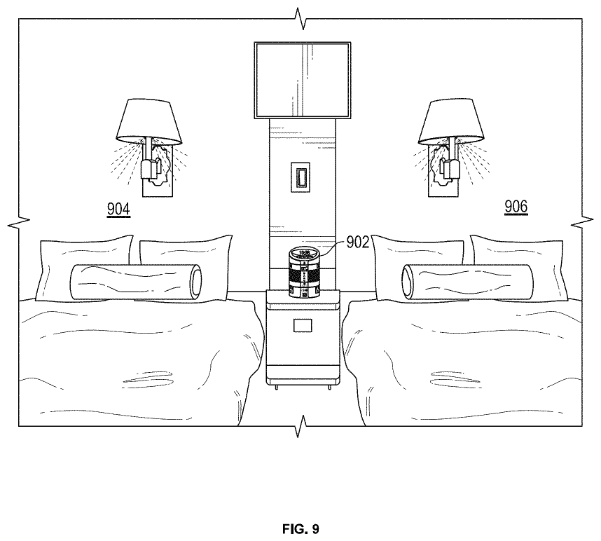

FIG. 10 is a schematic view of an alternative embodiment of an IOT control module disposed on a bedside table, illustrating a smart phone charging station on a top surface of the IOT control module.

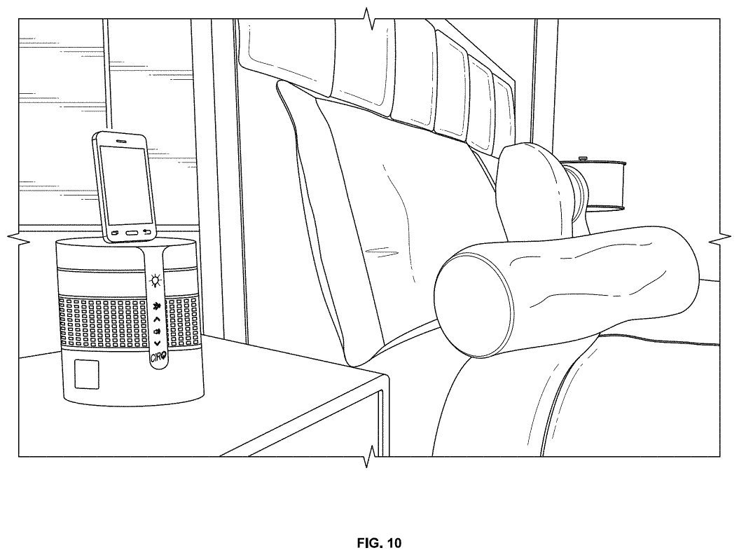

FIG. 11 is a schematic view of a front desk 1101 equipped with a base module 1102 configured to communicate with or embody a display 1104. In the illustrated embodiment, as a guest 1106 approaches a hotel employee 1108, the guest's location is tracked by the system, and the guest's name may be displayed on the screen 1104, or spoken to the employee through an ear piece 1110. In this way, the employee may address the guest by name using on real time location tracking data.

FIG. 12 is a schematic view of an alternative embodiment of a base module illustrating a self check-in and check-out module system allowing guests to perform self-registration, room upgrades and check-in into the property without having to directly interact with a property owner staff or employee, expediting their access to the purchased room. The illustrated embodiment includes a user interface, a display 1202, and a key card maker and credit card reader 1204.

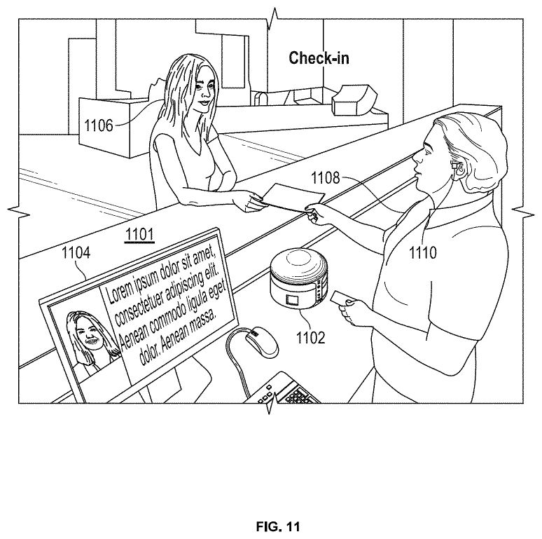

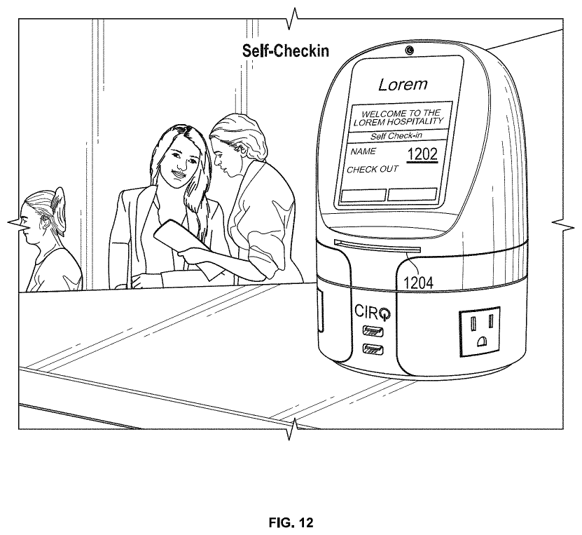

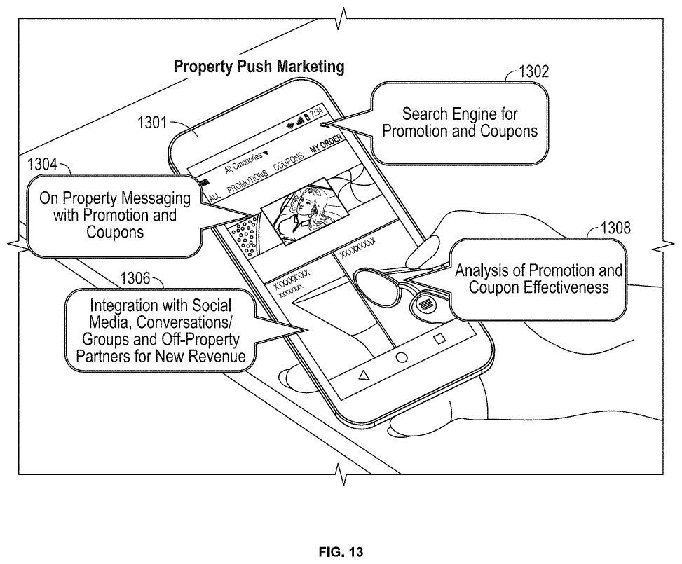

FIG. 13 is an exemplary guest smart phone 1301 running a mobile app configured to display a property specific search feature 1302 for services, searching promotions, upgrades, and incentives, a proprietary (on property) messaging portal 1304 for receiving notices, offers, promotions and messages, an integrated social media portal 1306, and an analytics portal 1308. Alternatively, the foregoing functionality may be hosted locally or remotely, without the need for the guest to download a mobile app.

Various embodiments of the present invention remote thermostatic control of an in-room heating, ventilation, and air conditioning (HVAC) unit such as a packaged terminal air conditioner (PTAC). PTACs are typically single, commercial grade, self-contained units installed through or inside a wall or window of a hotel guest room. A PTAC's compressor system both cools and heats. To cool, the units compressor pumps refrigerant to cool the coils which attracts heat and humidity which is then exhausted to the outside. To heat, this functionality is reversed. The refrigerant is used to heat the coils, and when air passes over it the unit pushes the heated air into the room. PTACs are larger than a typical through-the-wall air conditioner and can be wired controlled through the relay or wireless controlled via the in-room hub.

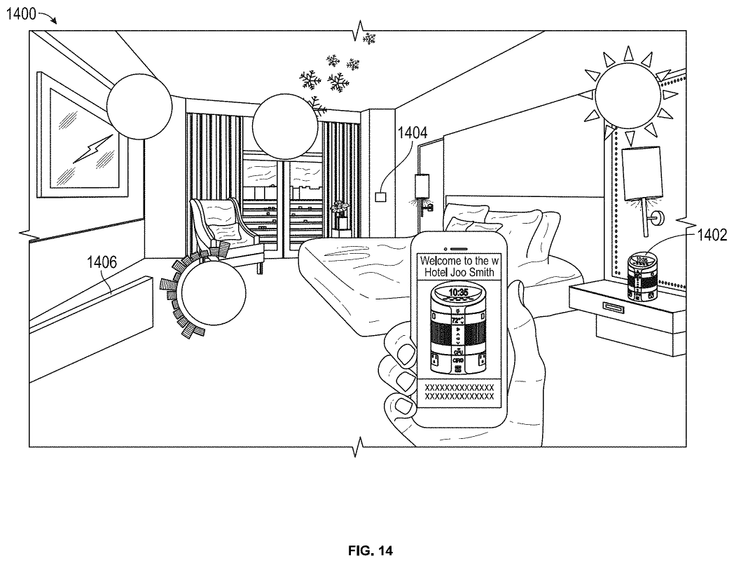

With continued reference to FIGS. 5-10 and also referring now to FIG. 14, a remote thermostatic control system 1400 includes an in-room IOT module 1402, a relay 1404 designed to replace a conventional wall-mounted thermostat (not shown), and an HVAC unit 1406 (e.g., a PTAC). In the illustrated embodiment, the IOT module 1402 includes a thermostat controller operable by the user to remotely (e.g., wirelessly) control the state of the relay 1404 which, in turn, operates the PTAC 1406 in much the same way (typically a wired connection) as the wall mounted thermostat previously did so before being replaced (or augmented) by the relay.

In an alternate embodiment, FIG. 15 depicts a remote thermostatic control system 1500 including an IOT module 1502 and other in-room connected array of sensors configured to communicate (e.g., wirelessly) with one or more secondary wireless modules 1504, 1505, and an HVAC unit 1506 (e.g., a PTAC). In the embodiment shown in FIG. 15, the IOT module 1502 includes a thermostat module which transmits (e.g., wirelessly) a desired temperature setting (e.g., set point) to one or both of the secondary wireless modules 1504, 1505 to thereby operate (e.g., wirelessly 1507) the PTAC 1506. In one embodiment, the user controls the PTAC 1506 using a handheld device 1504 (e.g., mobile phone, laptop, or other remote control device) which optionally displays a graphical user interface. In an alternate embodiment, the user may control the PTAC 1506 using a large screen display (e.g., computer monitor or television) which optionally displays a graphical user interface.

The embodiments described in conjunction with FIGS. 14 and 15 are particularly advantageous in that the temperature sensor associated with the thermostatic control system may reside within the bedside or table-top IOT module removed from the wall. In either case, the sensed temperature corresponds to the temperature proximate the hotel guest, particularly whilst the guest is sleeping. This allows the system to more precisely control the relevant temperature, ensuring thermal comfort while conserving electricity by avoiding unnecessarily heating or cooling regions of the guest room not occupied by the guest.

Referring now to FIG. 16, a distributed monitoring and sensing system 1600 includes an in-room IOT module 1602 coupled (e.g., wirelessly) to a plurality of distributed sensors 1604, 1606, 1608 equipped to monitor motion and/or temperature at a plurality of zones. For example, a first sensor 1604 may detect the temperature (or other environmental conditions such as smoke, carbon monoxide, brightness level, sound, and/or humidity) as well as the presence of or motion of people (or pets) in a region of the room remote from the bedroom. A second sensor 1606 may be configured to monitor one or more of the foregoing parameters proximate a balcony or window. A third sensor 1608 may be configured to monitor one or more of the foregoing parameters proximate a sitting area, an additional room, or other strategic location within the guest suite.

In accordance with the foregoing embodiments, by monitoring environmental parameters and the presence or motion of people at various locations within the living quarters, the system may precisely monitor and/or control energy and other resource consumption. By way of non-limiting example, the system may be configured to open or close window curtains or blinds in coordination with sunrise, sunset, and overcast conditions to thereby influence temperature control within the entire room or within discreet zones. Moreover, the system may optimize temperature, other environmental conditions, or the use of electronic devices as people migrate into and out of the living quarters or sub-zones thereof. The system will also allow for guest to migrate their personal preferences from property to property.

In accordance with further embodiments, the in-room IOT module and/or the underlying operating platform may include incentive, reward, or point based components configured to gamify energy conservation objectives. For example, the system may be configured to compile individual guest and/or aggregate data surrounding consumption of electricity, gas, cold water, hot water, towel and bed linen usage, and other consumables. By establishing usage targets or thresholds, hotel guests may earn loyalty credits or other redeemable points in a gamified context, while at the same time promoting “green” conservation policies.

In an embodiment, cellular data to and from the guest mobile device may be routed thru the CIRQ cloud 106 back to the hotel chain server 108 (See FIG. 1). Various use cases enabled by the system share the following features: i) a PaaS System; ii) an in-room IOT control hub module which communicates with the mobile app and various edge devices and hotel services (food, drinks); and iii) tracking of guest location in the CIRQ cloud. The integration of the in-room IOT network with guest tracking data gives rise to a vast array of novel features, use cases, and anecdotal attributes, including the following non-limiting examples.

In a typical swimming pool, beach, golf course, concert venue, or other resort environment having multiple potential guest locations (e.g., bar stools, tables, cabanas, chaise lounges, stadium seats), location awareness allows the server to walk a straight line to bring the correct drink or food order directly to the right guest.

Predictive analytics may be used in conjunction with tracking data. For example, if a guest returns to the guest room at the same time (e.g., 6:00 p.m.) several days in a row, the system may begin pre-cooling the room in anticipation of the guest returning, for example at 5:45 p.m. The system may be configured to fully cool the room when the guest actually enters the geo-fence surrounding the hotel property.

When a guest returns to a particular city, the system can recommend the same or similar restaurants based on previous visits to that city, or even monitor the guest's restaurant reviews (e.g., Yelp) to see how well the guest liked the food and/or venue, and make recommendations accordingly.

Targeted marketing can be in the form of a wrapper around newspaper left in the room or outside the room door. Alternatively, targeted marketing may take the form of text messages (e.g., SMS), notices and offers, or a short video displayed on the in-room television when turned on by the guest.

When incenting a guest to enable location services, for example in the context of a rewards program or an enhanced gamification opportunity, the system may be configured to offer enhanced (e.g. double) points or other incentives for qualified purchases if the location service is enabled, and further enhanced (e.g., tripled) points or other incentives if the guest allows ads to be pushed to their smart phone. Additionally the gamification feature may allow guests to receive additional incentives for limiting their power usage in the room (e.g., turning off lights, adjusting the temperature higher while away from the room, using towels/linens for more than one day, limiting water usage in the shower, faucet, and other water interfaces.

As an additional revenue source, the restaurants or other venues, for example through partnerships or by sponsoring targeted ads, may be required to pay the property owner (e.g., resort operator, hotel chain, time share aggregator) a percentage of payments for the privilege of pushing targeted ads to captive and presumably high income consumers (e.g., the members of a particular loyalty or rewards program) or guests that are using the mobile app for the first time which would make the guest feel more comfortable in the hotel and building loyalty for the hotel. Additionally, in return for presenting and redirecting the guest to off-property locations, restaurants and services, the hotel owner could receive a percentage of the transaction.

When location services detects that a guest is leaving the hotel property around dinner time, the system may be configured to push a contextually aware message to the guest's phone, such as a happy hour drink special at the hotel bar in an attempt to keep the guest on the property. If the guest accepts, the system can subsequently push an ad for a dinner special (e.g., a reduced price) at the hotel restaurant, for example if the guest remains in the bar more than 15 minutes.

Location awareness may be used to identify the name of guests as they approach the front desk, the bar, concierge, or any other venue, thereby allowing resort employees to address guests by name.

Location awareness may also be used to display guest names on a digital sign or other display (e.g., a welcome sign) as each guest approaches the sign. Moreover, location awareness may be used to determine which floor an elevator need to go to transport a guest to the correct room, effectively eliminating the need to press elevator buttons on premium floors.

Contextual awareness may involve the use of previous data to determine a particular guest's food allergies, liquor preferences (e.g., Bombay gin), and/or food preferences (e.g., gluten free options).

When incenting a guest to enable location services, for example in the context of a loyalty program, the system may be configured to award double points (or a 15% discount privilege) if the guest uses the app while on the property, and to award triple points (or a 30% discount privilege) for so long as the guest continues to enable the location service after leaving the property.

In an embodiment the guest can use the in-room module to control the local IOT devices even without downloading the app, but the guest can control the module with the mobile device if the guest downloads the app to the mobile device.

The system may be configured to link with Air B&B, Travelocity, or other travel related sites to gather a list of people going to a particular destination (e.g., San Diego) for a particular purpose (e.g., to stay at a timeshare) during a particular time period (e.g., next week), and send contextually aware targeted ads to the entire group; that is, since they are staying at a timeshare, the system presumes they are leisure travelers (as opposed to business) and can send targeted ads promoting a leisure attraction, such as SeaWorld.

Although preferred embodiments are described in the context of hotel room, those skilled in the art will appreciate that IOT control modules may be installed in any number of environments such as Air B&B rentals, condominium communities, and the like.

In an embodiment, the in-room IOT module may be configured to remotely control an access feature such as a door lock, where the guest can use the mobile app to configure access preferences, such as sending a unique code in an SMS message, or using tracking to unlock the door when the guest is within a predetermined distance (e.g., ten feet) from the door. After check out, the system can send a different code to the next guest, and yet another code to housekeeping personnel. The system can also provide security alerts advising the guest that someone entered the room such as room housekeeping, maintenance or property management.

In another embodiment the hub with integrated colored LED's can pulse or glow when the alarm goes off to slowly wake the guest and delivering a more pleasant awakening experience.

In another embodiment, the hotel facilities manager can remotely lock, unlock, check the locked status, or change the access code for IOT connected room door locks.

When incenting a guest to enable location services, the system may be configured to offer premium movie channels, Hulu-type streaming or music services, or complimentary nightclub passes (based on guest demographics). Additionally the hub may allow for streaming media from guests' personal mobile devices such as video and audio through the system and to other peripheral devices such as in room TV's.

In other embodiments the guest can select a desired perk (incentive) in exchange for enabling location services, where the perks are harvested from aggregate data (e.g., where do people in this age bracket or other demographic metric tend to dine, are they motivated by discounts, drink specials, enhanced reward points, iTunes credits).

The system may also use aggregate location data to dynamically allocate personnel and resources in real time. For example, as more guest go to the bar, pool, restaurant, or conference center, hotel management can allocate and dispatch service additional personnel as needed.

In another embodiment, the system can be configured to use tracking data to identify approaching guests, and to discretely speak the guest names into a hotel employee's earpiece to thereby allow the employee to address the guest by name.

In various embodiments, the mobile device communicates with the in-room IOT controller via Bluetooth or other protocol while the guest is in the room, but when the guest leaves the room the app can transition so that the mobile app send location data directly with the CIRQ cloud.

In various embodiments, the system may be configured to monitor the room with a motion sensor in addition to or in lieu of location tracking to determine when the room is vacant and the temperature can be adjusted or roll back to a preconfigured setting, fans and television turned off or lights dimmed. In addition, room occupancy detection and analysis allows the system to ignore incidental or transient occupancy (e.g., by housekeeping or administrative personnel) and to forego turning on the air conditioning when hotel staff are in the room but the guest is absent from the room.

In an embodiment, the in-room IOT control system includes a thermostat control module which may be disposed bedside or on a table inside the room. In this way, the guest may adjust the room temperature from their bed without having to get up. In addition, the thermostat control module may be configured to transmit a wireless control signal to a wall mounted relay receiver which, in turn, communicates the control signal to an IOT relay which interfaces with the building HVAC system.

Alternatively, the guest can adjust the room temperature using the mobile app to control the remote relay thermostat or adjust the temperature with the controls on the wall relay.

In an embodiment, as the guest is returning to the room but still outside the property geo-fence, the mobile app sends location data to the CIRQ cloud over cellular or Wi-Fi, whereupon the CIRQ cloud sends the data to the hotel property's server, which tells the in room device to anticipatorily adjust environmental controls; when the guest enters the room, the phone switches to Bluetooth communication with the in-room IOT control module.

In various embodiments, the motion sensor, array of sensors and tracking feature can coordinate to record a log of how much time the guest spends sleeping, at on property venues (e.g., business center, hotel bar), and at off property venues, and infer leisure and spending trends from aggregate location and occupancy data.

In summary, what has been described is an internet-of-things (IOT) robotic sterilization system for use in the prevention of diseases, e.g., Coronavirus Disease 2019 (COVID-19), caused by pathogens such as coronavirus SARS-CoV-2 and other pathogens present within an interior space is described. A robotic sterilization device is communicatively coupled to the IOT base module via an IOT network, and includes a misting system fluidically coupled to a liquid reservoir, a sensor module including plurality of sensors, a controller, and a locomotion system. The robotic sterilization device navigates a path within the interior space while creating a disinfecting mist with the misting system, and may coordinate with other IOT-connected devices, such as the HVAC system, to more efficaciously sanitize the interior space.

Systems of the present disclosure may be described in terms of functional and/or logical block components and various processing steps. It should be appreciated that such block components may be realized by any number of hardware, software, and/or firmware components configured to perform the specified functions. For example, an embodiment of the present disclosure may employ various integrated circuit components, e.g., memory elements, digital signal processing elements, logic elements, look-up tables, or the like, which may carry out a variety of functions under the control of one or more microprocessors or other control devices.

In addition, those skilled in the art will appreciate that embodiments of the present disclosure may be practiced in conjunction with any number of systems, and that the systems described herein are merely exemplary embodiments of the present disclosure. Further, the connecting lines shown in the various figures contained herein are intended to represent example functional relationships and/or physical couplings between the various elements. It should be noted that many alternative or additional functional relationships or physical connections may be present in an embodiment of the present disclosure.

As used herein, the terms “module” or “controller” refer to any hardware, software, firmware, electronic control component, processing logic, and/or processor device, individually or in any combination, including without limitation: application specific integrated circuits (ASICs), field-programmable gate-arrays (FPGAs), dedicated neural network devices (e.g., Google Tensor Processing Units), electronic circuits, processors (shared, dedicated, or group) configured to execute one or more software or firmware programs, a combinational logic circuit, and/or other suitable components that provide the described functionality.

As used herein, the word “exemplary” means “serving as an example, instance, or illustration.” Any implementation described herein as “exemplary” is not necessarily to be construed as preferred or advantageous over other implementations, nor is it intended to be construed as a model that must be literally duplicated.

While the foregoing detailed description will provide those skilled in the art with a convenient road map for implementing various embodiments of the invention, it should be appreciated that the particular embodiments described above are only examples, and are not intended to limit the scope, applicability, or configuration of the invention in any way. To the contrary, various changes may be made in the function and arrangement of elements described without departing from the scope of the invention.

1. An internet-of-things (IOT) robotic sterilization system for use in the prevention of microorganisms and diseases caused by pathogens present within an interior space, the system comprising:
an IOT base hub module with integrated occupancy sensors located within the interior space and communicatively coupled to an IOT network; and
a robotic sterilization device communicatively coupled to the IOT base module via the IOT network, the robotic sterilization device comprising a misting system fluidically coupled to a liquid reservoir source, a sensor module including plurality of sensors, a controller, and a locomotion system;
wherein the robotic sterilization device operates autonomously to navigate, using the sensor module, a path within the interior space while deploying a disinfecting mist with the misting system from a sterilization liquid provided within the liquid reservoir source.

2. The system of claim 1, wherein the sterilization liquid is selected to substantially eliminate the presence of coronavirus SARS-CoV-2 within the environment of the interior space.

3. The system of claim 1, wherein the sterilization liquid comprises hypochlorous acid (HOCl).

4. The system of claim 1, wherein the misting system includes an electrostatic misting device.

5. The system of claim 1, wherein the robotic sterilization device further includes a UV light.

6. The system of claim 1, wherein the IOT hub module is communicatively coupled to at least one occupancy sensor and to an HVAC component associated with the interior space, and is configured to perform at least one of: (1) disabling the HVAC component while creating the disinfecting mist and enabling to deliver conditioned air, and (2) UV lights located in the discharge of a variety of HVAC vents to further eliminate or reduce the aerosolized pathogens.

7. The system of claim 1, wherein the sensor module includes one or more sensors selected from the group consisting of accelerometers, magnetometers, lidar sensors, radar sensors, gyroscopic sensors, proximity sensors, optical sensors, sonar sensors, biosensors, thermal sensors, and location sensors.

8. The system of claim 1, wherein the robotic sterilization device includes a biosensor module configured to detect the presence of pathogens in the environment, and to activate the misting system in response to determining that the pathogen level within the environment is above a predetermined threshold.

9. The system of claim 1, wherein the biosensor module is provided within the interior space and is configured to detect the presence of pathogens in the environment; further wherein the robotic sterilization device activates the misting system in response to determining that the pathogen level within the environment is above a predetermined threshold.

10. The system of claim 1, further including a scent dispensing appliance configured to provide a burst of scent within the interior space after completion of the sterilization process.

11. The system of claim 1, wherein the misting system is configured to dispense the mist in a spray pattern selected from the group consisting of uni-directional, bi-directional, multi-level and omnidirectional.

12. The system of claim 11, wherein the uni-directional and bi-directional configurations cause a spray pattern that is perpendicular to a navigation sensor to thereby avoid occluding the navigation sensor.

13. A method of sterilizing an interior space with an internet-of-things (IOT) robotic system to prevent diseases caused by pathogens, reducing exposure to humans with the system comprising:
providing an IOT base module located within the interior space and communicatively coupling the IOT base to an IOT network; and
deploying, within the interior space, a robotic sterilization device communicatively coupled to the IOT base module via the IOT network, the robotic sterilization device comprising a misting system fluidically coupled to a liquid reservoir, a sensor module including plurality of sensors, a controller, and a locomotion system;
causing the robotic sterilization system to navigate, using the sensor module, a path within the interior space; and
creating a disinfecting mist with the misting system from a sterilization liquid provided within the liquid reservoir.

14. The method of claim 13, wherein the sterilization liquid comprises hypochlorous acid (HOCl).

15. The method of claim 13, wherein the sterilization liquid is selected to substantially eliminate the presence of coronavirus SARS-CoV-2 within the environment of the interior space.

16. The method of claim 13, wherein the system includes an electrostatic misting device.

17. The method of claim 13, wherein the robotic sterilization device further includes a UV light.

18. The method of claim 13, wherein the IOT hub module is communicatively coupled to an HVAC component associated with the interior space, and disables the HVAC component while the device is deploying disinfecting mist.

19. The method of claim 12, wherein the misting system is configured to dispense the mist in a spray pattern selected from the group consisting of uni-directional, bi-directional, multi-level and omnidirectional.

20. An internet-of-things (IOT) robotic sterilization system for use in the prevention of diseases caused by the presence of coronavirus SARS-CoV-2 within an interior space, the system comprising:
an IOT base module located within the interior space and communicatively coupled to an IOT network; and
a robotic sterilization device communicatively coupled to the IOT base module via the IOT network, the robotic sterilization device comprising an electrostatic misting or fogging system fluidically coupled to a liquid reservoir, a sensor module including plurality of sensors, a controller, and a locomotion system;
wherein the robotic sterilization device is configured to navigate, using the sensor module, a path within the interior space while creating a disinfecting mist with the misting system from a sterilization liquid comprising hypochlorous acid (HOCl) provided within the liquid reservoir;
wherein the IOT hub module is communicatively coupled to an HVAC component associated with the interior space, and is configured to disable the HVAC component while creating the disinfecting mist.
